(19) United States
(12) Patent Application Publication
(10) Pub. No.: US 2019/0358349 A1
(43) Pub. Date: Nov. 28, 2019

(54) METHODS FOR EVALUATION OF TREATMENT AND PROGRESSION OF NEUROLOGICAL DISORDERS

(71) Applicant: Parkinson's Institute, Mountain View, CA (US)

(72) Inventor: Carrolee BARLOW, Mountain View, CA (US)

(21) Appl. No.: 16/421,152

(22) Filed: May 23, 2019

Related U.S. Application Data
(60) Provisional application No. 62/676,083, filed on May 24, 2018.

(51) Int. Cl. A61K 49/00

(52) U.S. Cl. CPC A61K 49/0004

(57) ABSTRACT

Provided herein are methods related to the evaluation of treatment efficacy for neurological disorders. Also provided are methods for evaluating the progression of neurological disorders.

### CROSS-REFERENCE

This application claims the benefit of U.S. Provisional Application No. 62/676,083, filed May 24, 2018, which application is incorporated herein by reference in its entirety.

### BACKGROUND

Clinical trials for Parkinson's disease often rely on the Unified Parkinson's Disease Rating Scale (UPDRS) as the primary outcome measure. The UPDRS was designed to measure response to therapies that improve primarily the bradykinesia, rigidity and tremor symptoms of Parkinson's. In PD, an important physiological function is balance. Balance may be minimally influenced by dopaminergic therapies. Impairments of balance are clinically meaningful as they increase a person's risks for falls, leading to significant morbidity and mortality.

### BRIEF SUMMARY

Provided herein, in some embodiments, is a method of evaluating an efficacy of treatment for a neurological disorder comprising: (a) providing a subject suffering from a neurological disorder with a dopamine receptor stimulating therapy; (b) performing a Timed Up and Go test on the subject to obtain a first measurement; (c) providing the subject with an additional therapeutic; (d) performing a Timed Up and Go test on the subject to obtain a second measurement; and (e) determining the efficacy of the additional therapeutic based on the difference between the first measurement and the second measurement, wherein the efficacy of the therapeutic is determined independently from the efficacy of the dopamine receptor stimulating therapy.

In some embodiments, the first measurement is a measurement of time. In some embodiments, the second measurement is a measurement of time. In some embodiments, the additional therapeutic is determined to be effective if the second measurement is not increased relative to the first measurement. In some embodiments, the additional therapeutic is determined to be effective if the second measurement is reduced relative to the first measurement. In some embodiments, the additional therapeutic is determined to be effective if the second measurement is reduced by at least 5%, at least 10%, at least 15%, at least 20%, at least 25%, at least 30%, at least 35%, at least 40%, at least 45%, at least 50%, at least 60%, at least 70%, at least 80%, or at least 90% relative to the first measurement.

In some embodiments, the neurological disorder is multiple system atrophy or Parkinson's disease. In some embodiments, the neurological disorder comprises a movement disorder. In some embodiments, the additional therapeutic is a LRRK2 inhibitor, an anticholinergic therapeutic, an adenosine receptor antagonist, a glutamate receptor antagonist, a glutamate receptor activator, an adrenoceptor antagonist, or a serotonin receptor agonist. In some embodiments, the LRRK2 inhibitor comprises a small molecule, an siRNA, or an antibody. In some embodiments, the LRRK2 inhibitor is selected from the group consisting of DL201, DL151, and derivatives and analogs thereof. In some embodiments, the anticholinergic therapeutic is selected from the group consisting of trihexyphenidyl, benztropine, orphenadrine, procyclidine, biperiden, and derivatives and analogs thereof. In some embodiments, the adenosine receptor antagonist is selected from the group consisting of istradefylline, preladenant, tozadenant, vipadenant and derivatives and analogs thereof. In some embodiments, the glutamate receptor antagonist is selected from the group consisting of amantadine, dipraglurant, mavoglurant and derivatives and analogs thereof. In some embodiments, the glutamate receptor activator is selected from the group consisting of ADX88178 and derivatives and analogs thereof. In some embodiments, the adrenoceptor antagonist is selected from the group consisting of idazoxan, fipamezole, and derivatives and analogs thereof. In some embodiments, the serotonin receptor agonist is selected from the group consisting of sarizotan, piclozotan, and derivatives and analogs thereof. In some embodiments, the dopamine receptor stimulating therapy comprises L-dopa, levodopa, levodopa and carbidopa, a dopamine agonist, a monoamine oxidase B inhibitor, a catechol-O-methyltransferase inhibitor, or any combination thereof. In some embodiments, the monoamine oxidase B inhibitor is selected from the group consisting of selegiline, rasagiline, safinamide and derivatives and analogs thereof. In some embodiments, the dopamine agonist is selected from the group consisting of bromocriptine, pramipexole, ropinirole, rotigotine, apomorphine, and derivatives and analogs thereof. In some embodiments, the catechol-O-methyltransferase inhibitor is selected from the group consisting of tolcapone, entacapone, and derivatives and analogs thereof. In some embodiments, the subject is a human. In some embodiments, the subject is more than 30 years old, more than 40 years old, more than 50 years old, more than 60 years old, or more than 70 years old.

Provided herein, in some embodiments, is a method of evaluating an efficacy of treatment for a neurological disorder comprising: (a) providing a subject suffering from a neurological disorder with a dopamine receptor stimulating therapy and an additional therapeutic; (b) performing a Timed Up and Go test on the subject; and (c) determining the efficacy of the additional therapeutic based on the Timed Up and Go test, wherein the efficacy of the additional therapeutic is determined independently from the efficacy of the dopamine receptor stimulating therapy.

In some embodiments, the neurological disorder is multiple system atrophy or Parkinson's disease. In some embodiments, the neurological disorder comprises a movement disorder. In some embodiments, the additional therapeutic is a LRRK2 inhibitor, an anticholinergic therapeutic, an adenosine receptor antagonist, a glutamate receptor antagonist, a glutamate receptor activator, an adrenoceptor antagonist, or a serotonin receptor agonist. In some embodiments, the LRRK2 inhibitor comprises a small molecule, an siRNA, or an antibody. In some embodiments, the LRRK2 inhibitor is selected from the group consisting of DL201, DL151, and derivatives and analogs thereof. In some embodiments, the anticholinergic therapeutic is selected from the group consisting of trihexyphenidyl, benztropine, orphenadrine, procyclidine, biperiden, and derivatives and analogs thereof. In some embodiments, the adenosine receptor antagonist is selected from the group consisting of istradefylline, preladenant, tozadenant, vipadenant and derivatives and analogs thereof. In some embodiments, the glutamate receptor antagonist is selected from the group consisting of amantadine, dipraglurant, mavoglurant and derivatives and analogs thereof. In some embodiments, the glutamate receptor activator is selected from the group consisting of ADX88178 and derivatives and analogs thereof. In some embodiments, the adrenoceptor antagonist is selected from the group consisting of idazoxan, fipamezole, and derivatives and analogs thereof. In some embodiments, the serotonin receptor agonist is selected from the group consisting of sarizotan, piclozotan, and derivatives and analogs thereof. In some embodiments, the dopamine receptor stimulating therapy comprises L-dopa, levodopa, levodopa and carbidopa, a dopamine agonist, a monoamine oxidase B inhibitor, a catechol-O-methyltransferase inhibitor, or any combination thereof. In some embodiments, the monoamine oxidase B inhibitor is selected from the group consisting of selegiline, rasagiline, safinamide and derivatives and analogs thereof. In some embodiments, the dopamine agonist is selected from the group consisting of bromocriptine, pramipexole, ropinirole, rotigotine, apomorphine, and derivatives and analogs thereof. In some embodiments, the catechol-O-methyltransferase inhibitor is selected from the group consisting of tolcapone, entacapone, and derivatives and analogs thereof. In some embodiments, the subject is a human. In some embodiments, the subject is more than 30 years old, more than 40 years old, more than 50 years old, more than 60 years old, or more than 70 years old.

Provided herein, in some embodiments, is a method of treating a subject suffering from a neurological disorder comprising: (a) providing the subject with a dopamine receptor stimulating therapy; (b) performing a Timed Up and Go test on the subject to obtain a first measurement; (c) providing the subject with an additional therapeutic; (d) performing a Timed Up and Go test on the subject to obtain a second measurement; and (e) providing the subject with the additional therapeutic if the second measurement is not increased relative to the first measurement. In some embodiments, in (e), the subject is provided with the additional therapeutic if the second measurement is decreased relative to the first measurement.

In some embodiments, the neurological disorder is multiple system atrophy or Parkinson's disease. In some embodiments, the neurological disorder comprises a movement disorder. In some embodiments, the additional therapeutic is a LRRK2 inhibitor, an anticholinergic therapeutic, an adenosine receptor antagonist, a glutamate receptor antagonist, a glutamate receptor activator, an adrenoceptor antagonist, or a serotonin receptor agonist. In some embodiments, the LRRK2 inhibitor comprises a small molecule, an siRNA, or an antibody. In some embodiments, the LRRK2 inhibitor is selected from the group consisting of DL201, DL151, and derivatives and analogs thereof. In some embodiments, the anticholinergic therapeutic is selected from the group consisting of trihexyphenidyl, benztropine, orphenadrine, procyclidine, biperiden, and derivatives and analogs thereof. In some embodiments, the adenosine receptor antagonist is selected from the group consisting of istradefylline, preladenant, tozadenant, vipadenant and derivatives and analogs thereof. In some embodiments, the glutamate receptor antagonist is selected from the group consisting of amantadine, dipraglurant, mavoglurant and derivatives and analogs thereof. In some embodiments, the glutamate receptor activator is selected from the group consisting of ADX88178 and derivatives and analogs thereof. In some embodiments, the adrenoceptor antagonist is selected from the group consisting of idazoxan, fipamezole, and derivatives and analogs thereof. In some embodiments, the serotonin receptor agonist is selected from the group consisting of sarizotan, piclozotan, and derivatives and analogs thereof. In some embodiments, the dopamine receptor stimulating therapy comprises L-dopa, levodopa, levodopa and carbidopa, a dopamine agonist, a monoamine oxidase B inhibitor, a catechol-O-methyltransferase inhibitor, or any combination thereof. In some embodiments, the monoamine oxidase B inhibitor is selected from the group consisting of selegiline, rasagiline, safinamide and derivatives and analogs thereof. In some embodiments, the dopamine agonist is selected from the group consisting of bromocriptine, pramipexole, ropinirole, rotigotine, apomorphine, and derivatives and analogs thereof. In some embodiments, the catechol-O-methyltransferase inhibitor is selected from the group consisting of tolcapone, entacapone, and derivatives and analogs thereof. In some embodiments, the subject is a human. In some embodiments, the subject is more than 30 years old, more than 40 years old, more than 50 years old, more than 60 years old, or more than 70 years old.

Provided herein, in some embodiments, is a method of evaluating the progression of a neurological disorder comprising: (a) providing the subject with a dopamine receptor stimulating therapy and an additional therapeutic; (b) performing a Timed Up and Go test on the subject; and (c) evaluating the progression of the neurological disorder based on the Timed Up and Go test. In some embodiments, the method further comprises modifying the dose of the additional therapeutic or the dopamine receptor stimulating therapy based on the evaluating in (c). In some embodiments, the method further comprises further comprising providing the subject with an additional therapy based on the evaluating in (c).

In some embodiments, the neurological disorder is multiple system atrophy or Parkinson's disease. In some embodiments, the neurological disorder comprises a movement disorder. In some embodiments, the additional therapeutic is a LRRK2 inhibitor, an anticholinergic therapeutic, an adenosine receptor antagonist, a glutamate receptor antagonist, a glutamate receptor activator, an adrenoceptor antagonist, or a serotonin receptor agonist. In some embodiments, the LRRK2 inhibitor comprises a small molecule, an siRNA, or an antibody. In some embodiments, the LRRK2 inhibitor is selected from the group consisting of DL201, DL151, and derivatives and analogs thereof. In some embodiments, the anticholinergic therapeutic is selected from the group consisting of trihexyphenidyl, benztropine, orphenadrine, procyclidine, biperiden, and derivatives and analogs thereof. In some embodiments, the adenosine receptor antagonist is selected from the group consisting of istradefylline, preladenant, tozadenant, vipadenant and derivatives and analogs thereof. In some embodiments, the glutamate receptor antagonist is selected from the group consisting of amantadine, dipraglurant, mavoglurant and derivatives and analogs thereof. In some embodiments, the glutamate receptor activator is selected from the group consisting of ADX88178 and derivatives and analogs thereof. In some embodiments, the adrenoceptor antagonist is selected from the group consisting of idazoxan, fipamezole, and derivatives and analogs thereof. In some embodiments, the serotonin receptor agonist is selected from the group consisting of sarizotan, piclozotan, and derivatives and analogs thereof. In some embodiments, the dopamine receptor stimulating therapy comprises L-dopa, levodopa, levodopa and carbidopa, a dopamine agonist, a monoamine oxidase B inhibitor, a catechol-O-methyltransferase inhibitor, or any combination thereof. In some embodiments, the monoamine oxidase B inhibitor is selected from the group consisting of selegiline, rasagiline, safinamide and derivatives and analogs thereof. In some embodiments, the dopamine agonist is selected from the group consisting of bromocriptine, pramipexole, ropinirole, rotigotine, apomorphine, and derivatives and analogs thereof. In some embodiments, the catechol-O-methyltransferase inhibitor is selected from the group consisting of tolcapone, entacapone, and derivatives and analogs thereof. In some embodiments, the subject is a human. In some embodiments, the subject is more than 30 years old, more than 40 years old, more than 50 years old, more than 60 years old, or more than 70 years old.

Provided herein, in some embodiments, is a method of determining an optimum dosage level of a therapeutic for treatment of a neurological disorder in a subject suffering therefrom, comprising: (a) administering to the subject a first dosage level of a therapeutic agent for a number of doses at a dosing frequency; (b) performing a Timed Up and Go test to the subject to obtain a first measurement; (c) administering an increased dosage level of the therapeutic agent, relative to the first dosage level, for a number of doses at a dosing frequency; (d) performing a second Timed Up and Go test to the subject to obtain a second measurement; (e) determining an optimal dosage level from the two dosage levels administered, based on improvement in Timed Up and Go test and initiation of side effects. In some embodiments, the method further comprises repeating (b)-(e) until a maximum dosage level is achieved, no further improvement in test performance is detected, side effects outweigh benefits of treatment, or a combination thereof.

In some embodiments, the neurological disorder is multiple system atrophy or Parkinson's disease. In some embodiments, the neurological disorder comprises a movement disorder. In some embodiments, the additional therapeutic is a LRRK2 inhibitor, an anticholinergic therapeutic, an adenosine receptor antagonist, a glutamate receptor antagonist, a glutamate receptor activator, an adrenoceptor antagonist, or a serotonin receptor agonist. In some embodiments, the LRRK2 inhibitor comprises a small molecule, an siRNA, or an antibody. In some embodiments, the LRRK2 inhibitor is selected from the group consisting of DL201, DL151, and derivatives and analogs thereof. In some embodiments, the anticholinergic therapeutic is selected from the group consisting of trihexyphenidyl, benztropine, orphenadrine, procyclidine, biperiden, and derivatives and analogs thereof. In some embodiments, the adenosine receptor antagonist is selected from the group consisting of istradefylline, preladenant, tozadenant, vipadenant and derivatives and analogs thereof. In some embodiments, the glutamate receptor antagonist is selected from the group consisting of amantadine, dipraglurant, mavoglurant and derivatives and analogs thereof. In some embodiments, the glutamate receptor activator is selected from the group consisting of ADX88178 and derivatives and analogs thereof. In some embodiments, the adrenoceptor antagonist is selected from the group consisting of idazoxan, fipamezole, and derivatives and analogs thereof. In some embodiments, the serotonin receptor agonist is selected from the group consisting of sarizotan, piclozotan, and derivatives and analogs thereof. In some embodiments, the dopamine receptor stimulating therapy comprises L-dopa, levodopa, levodopa and carbidopa, a dopamine agonist, a monoamine oxidase B inhibitor, a catechol-O-methyltransferase inhibitor, or any combination thereof. In some embodiments, the monoamine oxidase B inhibitor is selected from the group consisting of selegiline, rasagiline, safinamide and derivatives and analogs thereof. In some embodiments, the dopamine agonist is selected from the group consisting of bromocriptine, pramipexole, ropinirole, rotigotine, apomorphine, and derivatives and analogs thereof. In some embodiments, the catechol-O-methyltransferase inhibitor is selected from the group consisting of tolcapone, entacapone, and derivatives and analogs thereof. In some embodiments, the subject is a human. In some embodiments, the subject is more than 30 years old, more than 40 years old, more than 50 years old, more than 60 years old, or more than 70 years old.

Provided herein, in some embodiments, is a method of determining an optimum dosage level of a therapeutic for treatment of a neurological disorder in a subject suffering therefrom, comprising: (a) administering to the subject a first dosage level of a therapeutic agent for a number of doses at a dosing frequency; (b) performing a Timed Up and Go test to the subject to obtain a first measurement; (c) administering a decreased dosage level of the therapeutic agent, relative to the first dosage level, for a number of doses at a dosing frequency; (d) performing a second Timed Up and Go test to the subject to obtain a second measurement; and (e) determining an optimal dosage level from the two dosage levels administered, based on change in Timed Up and Go test and decrease in side effects. In some embodiments, the method further comprises repeating (b)-(e) until a minimum dosage level is achieved, test performance deteriorates, side effects are minimized, or a combination thereof.

In some embodiments, the neurological disorder is multiple system atrophy or Parkinson's disease. In some embodiments, the neurological disorder comprises a movement disorder. In some embodiments, the additional therapeutic is a LRRK2 inhibitor, an anticholinergic therapeutic, an adenosine receptor antagonist, a glutamate receptor antagonist, a glutamate receptor activator, an adrenoceptor antagonist, or a serotonin receptor agonist. In some embodiments, the LRRK2 inhibitor comprises a small molecule, an siRNA, or an antibody. In some embodiments, the LRRK2 inhibitor is selected from the group consisting of DL201, DL151, and derivatives and analogs thereof. In some embodiments, the anticholinergic therapeutic is selected from the group consisting of trihexyphenidyl, benztropine, orphenadrine, procyclidine, biperiden, and derivatives and analogs thereof. In some embodiments, the adenosine receptor antagonist is selected from the group consisting of istradefylline, preladenant, tozadenant, vipadenant and derivatives and analogs thereof. In some embodiments, the glutamate receptor antagonist is selected from the group consisting of amantadine, dipraglurant, mavoglurant and derivatives and analogs thereof. In some embodiments, the glutamate receptor activator is selected from the group consisting of ADX88178 and derivatives and analogs thereof. In some embodiments, the adrenoceptor antagonist is selected from the group consisting of idazoxan, fipamezole, and derivatives and analogs thereof. In some embodiments, the serotonin receptor agonist is selected from the group consisting of sarizotan, piclozotan, and derivatives and analogs thereof. In some embodiments, the dopamine receptor stimulating therapy comprises L-dopa, levodopa, levodopa and carbidopa, a dopamine agonist, a monoamine oxidase B inhibitor, a catechol-O-methyltransferase inhibitor, or any combination thereof. In some embodiments, the monoamine oxidase B inhibitor is selected from the group consisting of selegiline, rasagiline, safinamide and derivatives and analogs thereof. In some embodiments, the dopamine agonist is selected from the group consisting of bromocriptine, pramipexole, ropinirole, rotigotine, apomorphine, and derivatives and analogs thereof. In some embodiments, the catechol-O-methyltransferase inhibitor is selected from the group consisting of tolcapone, entacapone, and derivatives and analogs thereof. In some embodiments, the subject is a human. In some embodiments, the subject is more than 30 years old, more than 40 years old, more than 50 years old, more than 60 years old, or more than 70 years old.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference in their entireties.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1 shows a flowchart for a method of determining the efficacy of a therapeutic using the Timed Up and Go test.

FIG. 2 shows a flowchart for a method of determining the efficacy of a therapeutic using the Timed Up and Go test.

FIG. 3 shows a flowchart for a method of evaluating the progression of a neurological disorder in a subject as described herein.

FIG. 4 shows a flowchart for evaluating the progression of a neurological disorder using the Timed Up and Go test.

FIG. 5 shows a flowchart for optimizing dosage amounts of a therapeutic using the Timed Up and Go test.

### DETAILED DESCRIPTION

### Overview

The UPDRS has been highly successful for measuring the therapeutic benefit of dopamine replacement therapies and other dopamine receptor stimulating therapies. However, the UPDRS may be insufficient to measure either disease progression or efficacy of additional interventions in patients who are on standard of care with adequate symptom management. Recognized herein is a need for methods of evaluating disease progression and therapeutic interventions in movement disorders, including those caused by neurological disorders (e.g., Parkinson's disease). There is a need for evaluation methods which can determine therapeutic efficacy and disease progression independent of the ongoing effects of a dopamine receptor stimulating therapy (e.g., L-dopa). Such evaluation may be useful for assessing clinical efficacy, informing treatment decisions, and determining the extent of disease progression.

Balance problems are common in the elderly and those with chronic diseases, including Parkinson's disease and other neurological disorders. As described herein, reliable tests of balance may be used to evaluate balance, gait, and other motor functions in neurological disorders, including multiple system atrophy (MSA) and Parkinson's disease, as a means of assessing disease progression and therapeutic efficacy. One such test is the Timed Up and Go test.

Described herein are methods for evaluating an efficacy of treatment for a neurological disorder (e.g., Parkinson's disease) comprising the use of the Timed Up and Go test, where the treatment efficacy is evaluated independently from that of dopamine receptor stimulating therapy. Measurements taken with a Timed Up and Go test from a subject being treated for a neurological disorder may be used to evaluate treatment efficacy. For example, a reduction in the measurements taken from a Timed Up and Go test from a subject may indicate efficacy of a treatment. These measurements may be useful in, for example, clinical trial evaluation. Additionally, measurements may be used to inform treatment decisions. For example, a Timed Up and Go test may demonstrate an improvement in a neurological disorder with a given therapeutic, thereby informing continued treatment with the therapeutic. A Timed Up and Go test may demonstrate no significant improvement in a neurological disorder with a given therapeutic, thereby informing cessation of treatment with the therapeutic (e.g., to eliminate harmful side effects).

Also described herein are methods for evaluating the progression of a neurological disorder (e.g., Parkinson's disease) comprising the use of the Timed Up and Go test to determine the degree of disease progression. Measurements taken with a Timed Up and Go test over time may serve to indicate the degree to which a neurological disorder is progressing, despite continued standard of care treatment (e.g., dopamine replacement therapy). Evaluation of disease progression may be useful in, for example, informing the use of additional treatment regimens. Indication that a disease is progressing using the methods disclosed herein may serve to indicate the need for an increase in the dose of a therapeutic and/or the use of one or more additional therapeutics.

Described herein are methods for evaluating an efficacy of treatment or a progression of a neurological disorder, wherein the subject exhibits one or more phenotypic presentations of the neurological disorder selected from the group consisting of: Parkinson's disease, Multisystem Lewy body disease (MLBD), parkinsonism, dementia with Lewy bodies (DLB), pure autonomic failure (PAF), Parkinson's disease dementia (PDD), multiple system atrophy, progressive supranuclear palsy, corticobasal degeneration, fronto-temporal dementia, Alzheimer's disease without Parkinson's disease, atypical parkinsonism, α-synuclein- or tau-related neuropathy, Lewy neurites or neuronal cell loss in substantia nigra, and α-synuclein-positive Lewy bodies. Also described are methods for evaluating an efficacy of treatment progression of a movement disorder caused by a neurological disorder, where the movement disorder is selected from the group consisting of: ataxia, Parkinson's disease and multiple system atrophy.

Also described herein are methods for evaluating an efficacy of treatment or a progression of a neurological disorder, wherein the subject has one or more mutations in one or more of the following genes: SNCA, LRRK2, GBA, LRRK2/GBA, PARK2, PINK1, 22q11 deletion, DNAJC13, VPS35, DJ-1, GCH1, ATXN2, C19orf12, PLA2G6, PANK2, DCTN1, TAF1, ATXN3, ATP13A2, FBX07, DNAJC6, SYNJ1, and SLC6A3.

### Definitions

The terminology used herein is for the purpose of describing particular cases only and is not intended to be limiting. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

The term “about” or “approximately” can mean within an acceptable error range for the particular value as determined by one of ordinary skill in the art, which will depend in part on how the value is measured or determined, i.e. the limitations of the measurement system. For example, “about” can mean within 1 or more than 1 standard deviation, per the practice in the art. Alternatively, “about” can mean a range of up to 20%, up to 10%, up to 5%, or up to 1% of a given value. Where particular values are described in the application and claims, unless otherwise stated the term “about” meaning within an acceptable error range for the particular value should be assumed. The term “about” has the meaning as commonly understood by one of ordinary skill in the art. In some embodiments, the term “about” refers to ±10%. In some embodiments, the term “about” refers to ±5%.

The term “symptom” refers to a subjective evidence of a disease, such as altered gait, as perceived by the patient. A “sign” refers to objective evidence of a disease as observed by a physician.

“Inhibition,” “treatment,” and “treating” are used interchangeably and refer to, for example, stasis of symptoms, prolongation of survival, partial or full amelioration of symptoms, and partial or full eradication of a condition, disease, or disorder.

A “subject,” “individual,” “host,” or “patient” refers to a living organism such as mammals. Examples of subjects include, but are not limited to, horses, cows, camels, sheep, pigs, goats, dogs, cats, rabbits, guinea pigs, rats, mice (e.g., humanized mice), gerbils, non-human primates (e.g., macaques), humans and the like, non-mammals, including, e.g., non-mammalian vertebrates, such as birds (e.g., chickens or ducks) fish (e.g., sharks) or frogs (e.g., Xenopus), and non-mammalian invertebrates, as well as transgenic species thereof. In certain aspects, a subject refers to a single organism (e.g., human).

“Treat” or “treatment” refers to a therapeutic treatment wherein the object is to eliminate or lessen symptoms.

### Timed Up and go Test

The methods disclosed herein may comprise the use of the Timed Up and Go test. A Timed Up and Go test may comprise four main parts. First, a subject may stand from a sitting position. The subject may be in a chair, on a bench, or otherwise seated in a sitting position. A subject may stand unassisted. A subject may stand with assistance, e.g., with assistance from arms from a chair. Second, a subject may walk forward a set distance. A distance may be about 3 meters. A distance may be about 10 feet. Third, a subject may turn and walk back to the starting position (e.g., back to a chair). A subject may walk unassisted. A subject may walk with assistance, e.g., with assistance from a walker, cane, etc. In some cases, a subject may walk without assistance from another person. Fourth, a subject may return to a sitting position (e.g., may sit down in a chair).

The time an individual takes to complete each part of a Timed Up and Go test may be measured. Measurements may be taken by one or more individuals. Measurements may be taken by one or more computers. The time an individual takes to complete one, two, three, or four parts of a Timed Up and Go test may be measured. In some cases, a measurement taken from a Timed Up and Go test may be used to evaluate the efficacy of a therapeutic. For example, a decrease in the time taken for a subject to complete the four parts of a Timed Up and Go test following treatment may indicate that a therapeutic is effective in treating a neurological disorder (e.g., Parkinson's disease). A Timed Up and Go test may be used to evaluate the efficacy of a therapeutic in treating Parkinson's disease, even in the presence of ongoing dopamine receptor stimulating therapy (e.g., L-dopa), thereby independently evaluating the efficacy of the therapeutic. In some cases, a measurement taken from a Timed Up and Go test may be used to evaluate the progression of a neurological disorder in a subject. For example, an increase in the time taken for a subject receiving continual dopamine receptor stimulating therapy (e.g., L-dopa) to complete the four parts of a Timed Up and Go test may indicate progression of Parkinson's disease.

A change in a measurement taken from a Timed Up and Go test may be useful in evaluating treatment efficacy. A reduction in a measurement of at least 5%, at least 10%, at least 15%, at least 20%, at least 25%, at least 30%, at least 35%, at least 40%, at least 45%, at least 50%, at least 60%, at least 70%, at least 80%, at least 90%, or greater may indicate the efficacy of a therapeutic. A reduction in a measurement of at most 5%, at most 10%, at most 15%, at most 20%, at most 25%, at most 30%, at most 35%, at most 40%, at most 45%, or at most 50%, at most 60%, at most 70%, at most 80%, or at most 90%, may indicate the efficacy of a therapeutic.

A change in a measurement taken from a Timed Up and Go test may be useful in evaluating disease progression. An increase in a measurement of about 5%, about 10%, about 15%, about 20%, about 25%, about 30%, about 35%, about 40%, about 45%, about 50%, about 60%, about 70%, about 80%, about 90%, or greater may indicate the progression of a neurological disorder (e.g., Parkinson's disease). An increase in a measurement of at least 5%, at least 10%, at least 15%, at least 20%, at least 25%, at least 30%, at least 35%, at least 40%, at least 45%, at least 50%, at least 60%, at least 70%, at least 80%, at least 90%, or greater may indicate the progression of a neurological disorder (e.g., Parkinson's disease). An increase in a measurement of at most 5%, at most 10%, at most 15%, at most 20%, at most 25%, at most 30%, at most 35%, at most 40%, at most 45%, or at most 50%, at most 60%, at most 70%, at most 80%, or at most 90%, may indicate the progression of a neurological disorder (e.g., Parkinson's disease).

### Methods of Evaluating Treatment Efficacy Using a Timed Up and go Test

Disclosed herein are methods for evaluating the treatment of a subject suffering from a neurological disorder (e.g., a movement disorder). In some cases, a dopamine receptor stimulating therapy may be provided to a subject suffering from a neurological disorder. Next, a Timed Up and Go test may be performed on the subject to obtain a first measurement. Next, the subject may be provided with an additional therapeutic. Next, a Timed Up and Go test may be performed on the subject to obtain a second measurement. The efficacy of the additional therapeutic may be determined based on the difference between the first measurement and the second measurement. The efficacy of the therapeutic may be determined independently from the efficacy of the dopamine receptor stimulating therapy.

A dopamine receptor stimulating therapy may comprise dopamine replacement therapy, a decarboxylase inhibitor, a dopamine agonist, a monoamine oxidase B inhibitor, a catechol-O-methyltransferase inhibitor, or a combination thereof. A dopamine receptor stimulating therapy may comprise levodopa, carbidopa, or a combination thereof. In some cases, a dopamine receptor stimulating therapy comprises levodopa and carbidopa. A measurement may be a measurement of time. A measurement may be a measurement in seconds. A measurement may be a time taken by a subject to perform one or more parts of the Timed Up and Go test. A measurement may be a time taken by a subject to perform all parts of the Timed Up and Go test. An additional therapeutic may be a LRRK2 inhibitor, an anticholinergic therapeutic, an adenosine receptor antagonist, a glutamate receptor antagonist, a glutamate receptor activator, an adrenoceptor antagonist, or a serotonin receptor agonist. In some cases, the additional therapeutic is a LRRK2 inhibitor. A LRRK2 inhibitor may comprise a small molecule, an siRNA, or an antibody. A LRRK2 inhibitor may be DL201, DL151, or derivatives and analogs thereof.

An additional therapeutic may be determined to be effective if the second measurement is not increased relative to the first measurement. An additional therapeutic may be determined to be effective if the second measurement is reduced relative to the first measurement. An additional therapeutic may be determined to be effective if the second measurement is reduced relative to the first measurement by at least 5%, at least 10%, at least 15%, at least 20%, at least 25%, at least 30%, at least 35%, at least 40%, at least 45%, at least 50%, at least 60%, at least 70%, at least 80%, or at least 90%. An additional therapeutic may be determined to be effective if the second measurement is reduced relative to the first measurement by at least 0.5 seconds, at least 1 second, at least 2 seconds, at least 3 seconds, at least 4 seconds, at least 5 seconds, at least 6 seconds, at least 7 seconds, at least 8 seconds, at least 9 seconds, or at least 10 seconds. An additional therapeutic may be determined to be effective if the second measurement is reduced relative to the first measurement by about 5%, about 10%, about 15%, about 20%, about 25%, about 30%, about 35%, about 40%, about 45%, about 50%, about 60%, about 70%, about 80%, or about 90%. An additional therapeutic may be determined to be effective if the second measurement is reduced relative to the first measurement by about 0.5 seconds, about 1 second, about 2 seconds, about 3 seconds, about 4 seconds, about 5 seconds, about 6 seconds, about 7 seconds, about 8 seconds, about 9 seconds, or about 10 seconds. An additional therapeutic may be determined to be ineffective if the second measurement is increased relative to the first measurement. A neurological disorder may be a movement disorder caused by a neurological disorder. A neurological disorder may be ataxia, Parkinson's disease, or MSA. In some cases, a neurological disorder is Parkinson's disease. In some cases, a neurological disorder is MSA.

In evaluating the efficacy of a therapeutic, a time taken by a subject to perform a Timed Up and Go test may be measured before and after treatment with the therapeutic. Comparing the results of such measurements may serve to determine the efficacy of the therapeutic. In particular, such results may determine the efficacy of the therapeutic independent of the efficacy of any dopamine receptor stimulating therapy that may also be provided to a subject. A therapeutic may be determined to be effective if the time a subject takes to perform a Timed Up and Go test is reduced following treatment with the therapeutic.

A Timed Up and Go test may be performed one or more times following treatment with a therapeutic. A subject may receive a therapeutic for a duration of time, and the Timed Up and Go test may be performed during the treatment duration to evaluate the efficacy of the therapeutic over time. In some cases, a therapeutic may be determined to be effective if the time a subject takes to perform a Timed Up and Go test remains about the same over time. In this case, a therapeutic may be effective at preventing or slowing the progression of a neurological disorder (e.g., Parkinson's disease) in the subject.

In some cases, a dopamine receptor stimulating therapy and an additional therapeutic may be provided to a subject suffering from a neurological disorder. Next, a Timed Up and Go test may be performed on the subject. Next, the efficacy of the additional therapeutic may be determined based on the Timed Up and Go test. The efficacy of the therapeutic may be determined independently from the efficacy of the dopamine receptor stimulating therapy.

A dopamine receptor stimulating therapy may comprise dopamine replacement therapy, a decarboxylase inhibitor, a dopamine agonist, a monoamine oxidase B inhibitor, a catechol-O-methyltransferase inhibitor, or a combination thereof. A dopamine receptor stimulating therapy may comprise levodopa, carbidopa, or a combination thereof. In some cases, a dopamine receptor stimulating therapy comprises levodopa and carbidopa. An additional therapeutic may be a LRRK2 inhibitor, an anticholinergic therapeutic, an adenosine receptor antagonist, a glutamate receptor antagonist, a glutamate receptor activator, an adrenoceptor antagonist, or a serotonin receptor agonist. In some cases, the additional therapeutic is a LRRK2 inhibitor. A LRRK2 inhibitor may comprise a small molecule, an siRNA, or an antibody. A LRRK2 inhibitor may be DL201, DL151, or derivatives and analogs thereof. A neurological disorder may be a movement disorder caused by a neurological disorder. A neurological disorder may be ataxia, Parkinson's disease, or MSA. In some cases, a neurological disorder is Parkinson's disease. In some cases, a neurological disorder is MSA.

The time taken for a subject receiving a therapeutic to perform a Timed Up and Go test may be compared to a given value (e.g., a “reference value”) to determine the efficacy of a therapeutic. A reference value may be an average time taken to complete a Timed Up and Go test by individuals with a given neurological condition (e.g., Parkinson's disease). An average time taken to complete a Timed Up and Go test by individuals with Parkinson's disease may be about 5 seconds, about 6 seconds, about 7 seconds, about 8 seconds, about 9 seconds, about 10 seconds, about 11 seconds, about 12 seconds, about 13 seconds, about 14 seconds, about 15 seconds, about 16 seconds, about 17 seconds, about 18 seconds, about 19 seconds, about 20 seconds, or about 21 seconds, or more. An appropriate reference value for use in evaluating the efficacy of a therapeutic may be determined based on one or more details about a subject (e.g., age, disease onset, disease phase, etc). A therapeutic may be determined to be effective if, following treatment, the time taken by a subject to complete a Timed Up and Go test is smaller than a reference value. A therapeutic may be determined to be effective if the time taken is about 5%, about 10%, about 20%, about 30%, about 40%, about 40%, about 50%, about 60%, about 70%, about 80%, or about 90% lower than a reference value. A therapeutic may be determined to be effective if the time taken is at least 5%, at least 10%, at least 20%, at least 30%, at least 40%, at least 50% lower, at least 60%, at least 70%, at least 80%, or at least 90% lower than a reference value. A therapeutic may be determined to be effective if the time taken is at most 5%, at most 10%, at most 20%, at most 30%, at most 40%, at most 50%, at most 60%, at most 70%, at most 80%, or at most 90% lower than a reference value.

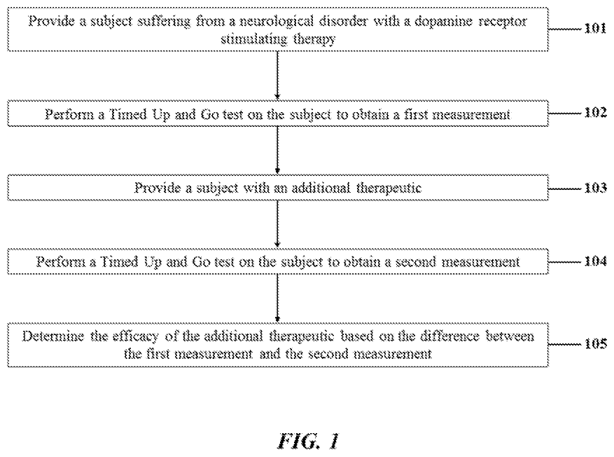

FIG. 1 shows an example method for evaluating the efficacy of a therapeutic in treating a neurological disorder. First, in operation 101, a dopamine receptor stimulating therapy (e.g., L-dopa) is provided to a subject suffering from a neurological disorder. In operation 102, a Timed Up and Go test is performed on the subject to obtain a first measurement. The first measurement may be a time taken by the subject to perform all the parts of a Timed Up and Go test. In operation 103, the subject is provided with an additional therapeutic. The additional therapeutic may be provided together with continued dopamine receptor stimulating therapy. In operation 104, a second Timed Up and Go test is performed on the subject to obtain a second measurement. The second measurement may be a time taken by the subject to perform all the parts of a Timed Up and Go test. Finally, in operation 105, the second measurement is compared to the first measurement, and the difference between the measurements is used to determine the efficacy of the additional therapeutic. For example, a decrease in the time taken by the subject to perform all the parts of the Timed Up and Go test in operation 104 relative to the time taken in operation 102 (e.g., a decrease of at least 5%, at least 10%, at least 20%, at least 30%, at least 40%, at least 50%, at least 60%, at least 70%, at least 80%, or at least 90%) may indicate that the additional therapeutic is effective in treating the neurological disorder.

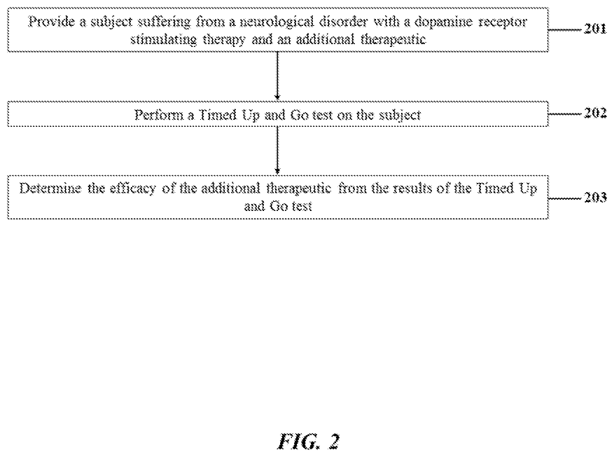

FIG. 2 shows another example method for evaluating efficacy of a therapeutic in treating a neurological disorder. In this example, first, in operation 201, a dopamine receptor stimulating therapy (e.g., L-dopa) and an additional therapeutic are provided to a subject suffering from a neurological disorder. In operation 202, a Timed Up and Go test is performed on the subject. Finally, in operation 203, the results of the Timed Up and Go test are used to determine the efficacy of the additional therapeutic. For example, the time taken to perform the steps of the Timed Up and Go test may be significantly lower (e.g., at least 5%, at least 10%, at least 20%, at least 30%, at least 40%, at least 50% lower, at least 60% lower, at least 70% lower, at least 80% lower, or at least 90% lower) than a given value, thereby indicating the efficacy of the additional therapeutic independent of the effects of the dopamine receptor stimulating therapy.

### Methods of Evaluating Disease Progression Using a Timed Up and go Test

Provided herein, in some aspects, are methods for evaluating the progression of a neurological disorder in a subject. In some cases, a dopamine receptor stimulating therapy and an additional therapeutic may be provided to a subject suffering from a neurological disorder. Next, a Timed Up and Go test may be performed on the subject. Next, the progression of the neurological disorder may be evaluated based on the Timed Up and Go test. The progression of the neurological disorder may be evaluated independent of the effects of the dopamine receptor stimulating therapy.

A dopamine receptor stimulating therapy may comprise dopamine replacement therapy (e.g., L-dopa, levodopa), a decarboxylase inhibitor (e.g., carbidopa), a dopamine agonist, a monoamine oxidase B inhibitor, a catechol-O-methyltransferase inhibitor, or a combination thereof. A dopamine receptor stimulating therapy may comprise levodopa, carbidopa, or a combination thereof. In some cases, a dopamine receptor stimulating therapy comprises levodopa and carbidopa. An additional therapeutic may be a LRRK2 inhibitor, an anticholinergic therapeutic, an adenosine receptor antagonist, a glutamate receptor antagonist, a glutamate receptor activator, an adrenoceptor antagonist, or a serotonin receptor agonist. In some cases, the additional therapeutic is a LRRK2 inhibitor. A LRRK2 inhibitor may comprise a small molecule, an siRNA, or an antibody. A neurological disorder may be a movement disorder caused by a neurological disorder. A neurological disorder may be ataxia, Parkinson's disease, or MSA. In some cases, a neurological disorder is Parkinson's disease. In some cases, a neurological disorder is MSA.

Evaluating the progression of a disorder based on a Timed Up and Go test may comprise determining the time taken for a subject to perform the steps of the Timed Up and Go test. The time taken for a subject to perform the steps of the Timed Up and Go test may significantly increase (e.g., increase by at least 5%, at least 10%, at least 20%, at least 30%, at least 40%, at least 50%, at least 60%, at least 70%, at least 80%, or at least 90%) over time, thereby indicating progression of a neurological disorder. The rate at which the time taken for a subject to perform the steps of the Timed Up and Go test increases over time may correlate with the rate of disease progression in the subject.

Certain methods of diagnosing a subject suffering from movement disorders caused by neurological disorders (e.g., Parkinson's disease, MSA) may be insufficient for evaluating disease progression in the presence of ongoing dopamine receptor stimulating therapy (e.g., L-dopa). Use of a Timed Up and Go test may enable the evaluation of the progression of such a disorder, even in the presence of ongoing maintenance therapy. Evaluating disease progression using the disclosed methods may serve to inform ongoing treatment decisions for a subject. For example, a Timed Up and Go test may be used to evaluate the progression of Parkinson's disease in a subject, thereby determining that a subject with Parkinson's disease is experiencing disease progression. Disease progression may be indicated, for example, by a subject's increase in time to complete a Timed Up and Go test. In this case, a treatment decision may be made, such as, for example, modifying the type of therapeutic (e.g., modification from one different dopamine receptor stimulating therapy to another), modifying the dose of therapeutic (e.g., increasing the dose), performing an additional therapy (e.g., performing deep brain stimulation), or enrolling a subject in clinical trial. In another example, a Timed Up and Go test may be used to evaluate the progression of Parkinson's disease in a subject, thereby determining that a subject with Parkinson's disease is not experiencing disease progression. A subject may be determined not to experience disease progression, for example, by lowering their time to complete a Timed Up and Go test. In this case, a treatment decision may be made, such as decreasing the dose of a therapeutic, for example, to alleviate side effects.

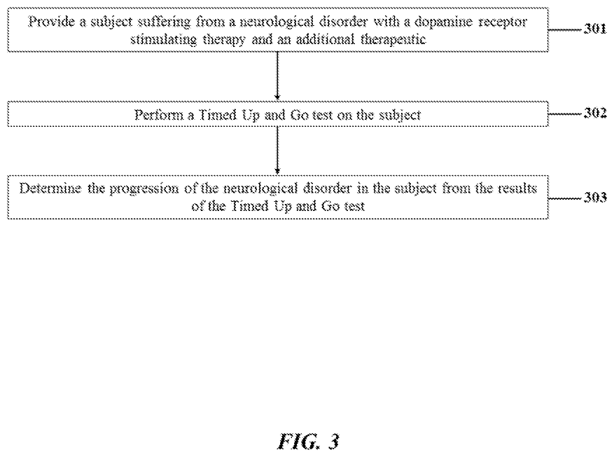

FIG. 3 shows an example method for evaluating the progression of a neurological disorder in a subject. In this example, in operation 301, a dopamine receptor stimulating therapy (e.g., L-dopa) and an additional therapeutic are provided to a subject suffering from a neurological disorder. In operation 302, a Timed Up and Go test is performed on the subject. Finally, in operation 303, the results of the Timed Up and Go test are used to determine the progression of the neurological disorder in the subject. Operations 301-303 may be repeated one or more times at intervals throughout the treatment duration of a subject.

### Methods of Treating a Neurological Disorder

Provided herein, in some aspects, are methods of treating or effecting prophylaxis of a neurological disease or disorder, for example Parkinson's disease or MSA. In some cases, a dopamine receptor stimulating therapy may be provided to a subject suffering from a neurological disorder. Next, a Timed Up and Go test may be performed on the subject to obtain a first measurement. Next, the subject may be provided with an additional therapeutic. Next, a Timed Up and Go test may be performed on the subject to obtain a second measurement. Next, a subject may be provided with the additional therapeutic if the second measurement is not increased relative to the first measurement. A subject may be provided with the additional therapeutic if the second measurement is reduced relative to the first measurement.

A dopamine receptor stimulating therapy may comprise dopamine replacement therapy, a decarboxylase inhibitor, a dopamine agonist, a monoamine oxidase B inhibitor, a catechol-O-methyltransferase inhibitor, or a combination thereof. A dopamine receptor stimulating therapy may comprise levodopa, carbidopa, or a combination thereof. In some cases, a dopamine receptor stimulating therapy comprises levodopa and carbidopa. A measurement may be a measurement of time. A measurement may be a measurement in seconds. A measurement may be a time taken by a subject to perform one or more parts of the Timed Up and Go test. A measurement may be a time taken by a subject to perform all parts of the Timed Up and Go test. An additional therapeutic may be a LRRK2 inhibitor, an anticholinergic therapeutic, an adenosine receptor antagonist, a glutamate receptor antagonist, a glutamate receptor activator, an adrenoceptor antagonist, or a serotonin receptor agonist. In some cases, the additional therapeutic is a LRRK2 inhibitor. A LRRK2 inhibitor may comprise a small molecule, an siRNA, or an antibody. A LRRK2 inhibitor may be DL201, DL151, or derivatives and analogs thereof.

An additional therapeutic may be provided if the second measurement is not increased relative to the first measurement. An additional therapeutic may be provided if the second measurement is reduced relative to the first measurement. An additional therapeutic may be provided if the second measurement is reduced relative to the first measurement by at least 5%, at least 10%, at least 15%, at least 20%, at least 25%, at least 30%, at least 35%, at least 40%, at least 45%, at least 50%, at least 60%, at least 70%, at least 80%, or at least 90%. An additional therapeutic may be provided if the second measurement is reduced relative to the first measurement by at least 0.5 seconds, at least 1 second, at least 2 seconds, at least 2 seconds, at least 3 seconds, at least 4 seconds, at least 5 seconds, at least 6 seconds, at least 7 seconds, at least 8 seconds, at least 9 seconds, or at least 10 seconds. An additional therapeutic may be provided if the second measurement is reduced relative to the first measurement by about 5%, about 10%, about 15%, about 20%, about 25%, about 30%, about 35%, about 40%, about 45%, about 50%, about 60%, about 70%, about 80%, or about 90%. An additional therapeutic may be provided if the second measurement is reduced relative to the first measurement by about 0.5 seconds, about 1 second, about 2 seconds, about 2 seconds, about 3 seconds, about 4 seconds, about 5 seconds, about 6 seconds, about 7 seconds, about 8 seconds, about 9 seconds, or about 10 seconds. An additional therapeutic may not be provided if the second measurement is increased relative to the first measurement. A neurological disorder may be a movement disorder caused by a neurological disorder. A neurological disorder may be ataxia, Parkinson's disease, or MSA. In some cases, a neurological disorder is Parkinson's disease. In some cases, a neurological disorder is MSA.

In some aspects, a Timed Up and Go test may be used to determine whether to provide a subject with a therapeutic. For example, a subject suffering from Parkinson's disease may be receiving ongoing maintenance therapy (e.g., dopamine receptor stimulating therapy, such as L-dopa). In this case, a Timed Up and Go test may be used to determine whether to provide the subject with an additional therapeutic (e.g., a LRRK2 inhibitor) by comparing the time taken to perform the steps of a Timed Up and Go test with a reference value. The time taken for a subject to complete the Timed Up and Go test may be greater than a reference value, thereby informing a decision to provide the subject with an additional therapeutic. The time taken for a subject to complete the Timed Up and Go test may be less than or about equal to a reference value, thereby informing a decision to refrain from providing the subject with an additional therapeutic.

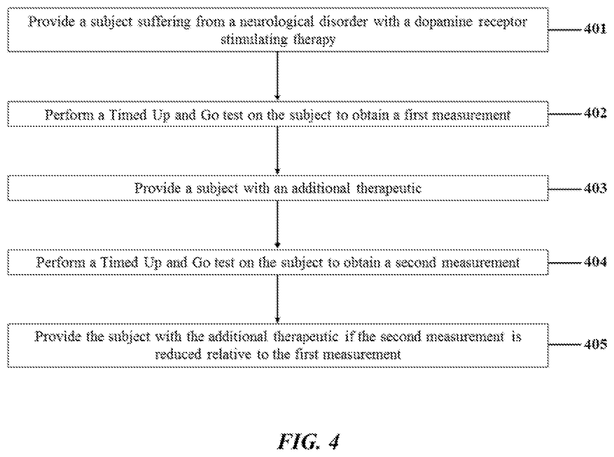

FIG. 4 shows an example method for treating a subject suffering from a neurological disorder. In this example, first, in operation 401, a dopamine receptor stimulating therapy (e.g., L-dopa) is provided to a subject suffering from a neurological disorder. In operation 402, a Timed Up and Go test is performed on the subject to obtain a first measurement. The first measurement may be a time taken by the subject to perform all the parts of a Timed Up and Go test. In operation 403, the subject is provided with an additional therapeutic. The additional therapeutic may be provided together with continued dopamine receptor stimulating therapy. In operation 404, a second Timed Up and Go test is performed on the subject to obtain a second measurement. The second measurement may be a time taken by the subject to perform all the parts of a Timed Up and Go test. Finally, in operation 405, the additional therapeutic is again provided to the subject if the second measurement is reduced (e.g., reduced by at least 5%, at least 10%, at least 20%, at least 30%, at least 40%, at least 50%, at least 60%, at least 70%, at least 80%, or at least 90%) relative to the first measurement. In some cases, if the second measurement is not reduced compared to the first measurement, the additional therapeutic is not provided to the subject.

### Dosage Optimization

Dosage of a therapeutic used in treating a neurological disorder may be optimized using methods described herein. In some cases, a subject may be administered a first dosage level of a therapeutic agent for a number of doses at a dosing frequency. The subject may then be administered a Timed Up and Go test to obtain a first measurement. Next, the subject may be administered an increased dosage level, relative to the first dosage level, of the therapeutic agent for a number of doses at a dosing frequency, followed by a second Timed Up and Go test to obtain a second measurement. Next, an optimal dosage level may be selected from the two dosage levels administered, based on improvement in Timed Up and Go test and initiation of, if any, side effects. An improvement in a Timed Up and Go test may be a reduction in the time taken to complete all steps of a Timed Up and Go test. Iterations of increasing dosage level and Timed Up and Go testing may be administered until a maximum dosage level is achieved, no further improvement in test performance is detected, or side effects outweigh benefits of treatment.

In some cases, a subject suffering from a neurological disorder may be administered an initial dosage level of a therapeutic agent for a number of doses at a dosing frequency, followed by a Timed Up and Go test. Next, the subject may be administered a decreased dosage level of the therapeutic agent for a number of doses at a dosing frequency, followed by a second Timed Up and Go test. An optimal dosage level may be selected from the two dosages levels administered, based on change in test performance and decrease in side effects, if any. Iterations of decreasing dosage level and Timed Up and Go test may be administered until a minimum dosage level is achieved, test performance deteriorates, or side effects are minimized. Deterioration of test performance may be an increase in the time taken for a subject to complete the steps of a Timed Up and Go test.

A therapeutic agent may be a LRRK2 inhibitor, an anticholinergic therapeutic, an adenosine receptor antagonist, a glutamate receptor antagonist, a glutamate receptor activator, an adrenoceptor antagonist, or a serotonin receptor agonist. In some cases, the additional therapeutic is a LRRK2 inhibitor. A LRRK2 inhibitor may comprise a small molecule, an siRNA, or an antibody. A LRRK2 inhibitor may be DL201, DL151, or derivatives and analogs thereof. A neurological disorder may be a movement disorder caused by a neurological disorder. A neurological disorder may be ataxia, Parkinson's disease, or MSA. In some cases, a neurological disorder is Parkinson's disease. In some cases, a neurological disorder is MSA.

A subject may be administered a first dosage level of a therapeutic agent which is below the recommended dosage. A subject may be administered an initial dosage which is the maximum recommended dose. A dosage level may be about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 30 mg, about 35 mg, about 40 mg, about 45 mg, or about 50 mg. The number of doses can be 1, 2, 3, 4, 5, 6, 7, 8, 9, 10, 12, 14, 16, 18, 20, 25, 30, 35, 40, 50, or 60 doses. The dosing frequency can be one hour, 2 hours, 3 hours, 4 hours, 5 hours, 6 hours, 7 hours, 8 hours, 9 hours, 10 hours, 11 hours, 12 hours, 14 hours, 16 hours, 18 hours, 20 hours, 22 hours, one day, 2 days, 3 days, 4 days, 5 days 6 days, one week, 2 weeks, 3 weeks, 4 weeks, one month, 2 months, 3 months, 4 months, 5 months, 6 months, or one year. Dosage level may be increased by about 5%, about 10%, about 20%, about 30%, about 40%, about 50%, about 60%, about 70%, about 80%, about 90%, or about 100%. Dosage level may be decreased by about 5%, about 10%, about 20%, about 30%, about 40%, about 50%, about 60%, about 70%, about 80%, about 90%, or about 100%.

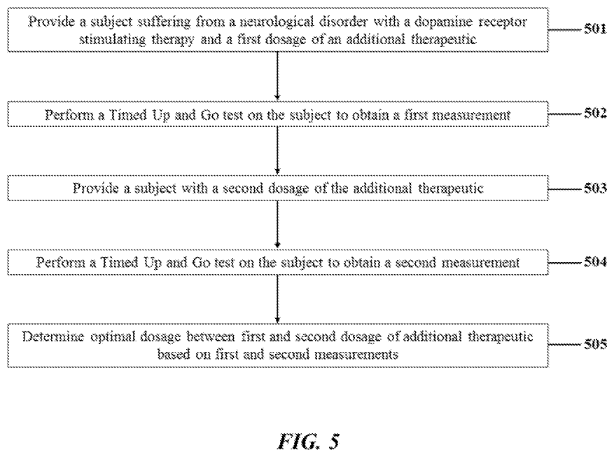

An example of dosage optimization as disclosed herein is shown in FIG. 5. In this example, a subject with a neurological disorder is provided with a dopamine receptor stimulating therapy (e.g., L-dopa) and a first dosage of an additional therapeutic in operation 501. The subject performs a Timed Up and Go test to obtain a first measurement in operation 502. The subject is provided a second dosage of the additional therapeutic in operation 503. The subject performs a Timed Up and Go test to obtain a second measurement in operation 504. An optimal response between first dosage and second dosage is determined based on an improvement or deterioration in time measurement between the first measurement and the second measurement in operation 505. Iterations of operations 503-505 may be performed to “fine tune” optimal dosage levels. Providing a subject with a second dosage in operation 503 is performed at an appropriate dosage interval following providing the first dosage in operation 501 to avoid stacking doses of therapeutic in the subject. For example, a subject may be provided with the second therapeutic at a second dosage in operation 503 at about t_{1/2}, about 2×t_{1/2}, about 3×t_{1/2}, about 4×t_{1/2}, about 5×t_{1/2}, or more after providing the additional therapeutic at a first dosage in operation 501.

### Treatment and Therapy

### Dopamine Receptor Stimulating Therapy

As used herein, a “dopamine receptor stimulating therapy” may describe a treatment for a neurodegenerative disorder which increases the activity of one or more dopamine receptors in a subject. A dopamine receptor stimulating therapy may be dopamine replacement therapy comprising dopamine or a derivative, analog, or prodrug thereof (e.g., L-dopa, levodopa). A dopamine receptor stimulating therapy may comprise a decarboxylase inhibitor. A decarboxylase inhibitor may reduce the rate at which dopamine or a derivative, analog, or prodrug thereof is metabolized by a subject. A decarboxylase inhibitor may be carbidopa. A dopamine receptor stimulating therapy may comprise a dopamine agonist. A dopamine agonist may comprise bromocriptine, pramipexole, ropinirole, rotigotine, apomorphine, or derivatives and analogs thereof. A dopamine receptor stimulating therapy may comprise a monoamine oxidase B inhibitor. A monoamine oxidase B inhibitor may be selegiline, rasagiline, safinamide or derivatives and analogs thereof. A dopamine receptor stimulating therapy may comprise a catechol-O-methyltransferase inhibitor. A catechol-O-methyltransferase inhibitor may be tolcapone, entacapone, or derivatives and analogs thereof.

### LRRK2 Inhibitors

A LRRK2 inhibitor may be administered to treat neurodegenerative diseases. A LRRK2 inhibitor may be administered to treat neurological disorders (e.g., Parkinson's disease, MSA). Efficacy of a LRRK2 inhibitor may be evaluated using the methods disclosed herein. A LRRK2 inhibitor may be a small molecule (e.g., a kinase inhibitor), an siRNA, or an antibody. A LRRK2 inhibitor may be a staurosporine derivative, a naleimide derivative, an indolinone derivative, a quinoline derivative, an anthracene derivative, a phenanthrene derivative, or a pyrimidine derivative. A LRRK2 inhibitor may be at least one of DNL201, DNL151, PD-98059, U-0126, SB-203580, H-7, H-9, Staurosporine, AG-494, AG-825, Lavendustin A, RG-14620, Tyrphostin 23, Tyrphostin 25, Tyrphostin 46, Tyrphostin 47, Tyrphostin 51, Tyrphostin 1, Tyrphostin AG1288, Tyrphostin AG1478, Tyrphostin AG1295, Tyrphostin 9, hydroxy-2-naphthalene ethylphosphoric acid, Damnacanthal, piceatannol, PP1, AG-490, AG-126, AG-370, AG-879, LY294002, wortmannin, GP109203X, hypericin, Ro31-8220, Sphingosine, H-89, H-8, HA-1004, HA-1077, 2-hydroxy-5-(2,5-dihydroxybenzyl-amino) benzoic acid, HG-10-102-01, KN-62, KN-93, ML-7, ML-9, MLi-2, 2-aminopurine, N9-isopropyl olomoucine, Olomoucine, iso-olomoucine, roscovitine, 5-iodo-tubercidin, LFM-A13, SB-202190, PP2, ZM336372, SU4312, AG-1296, GW5074, palmitoyl-DL-carnitine Cl, rottlerin, genistein, daidzein, erbstatin analog, quercetin dihydrate, SU1498, ZM449829, BAY11-7082, 5,6-dichloro-1-beta-D-ribofutanosyl-benzimidiazole, 2,2′,3,3′,4,4′-hexahydroxy-1,1′-biphenyl-6,6′-dimenthanol dimethyl ester, SP600126, Indirubin, Indirubin-3-monooxine, cantharidic acid, cantharidin, endothall, benzyl-phosphoric acid, L-p-bromo-tetraamisole oxalate, RK-682, RWJ-60475, levarmisole HCl, tetramisole HCl, cypermethrin, deltamethrin, fenvaierate, Tyrphostin 8, Cinngel, LDN-22684, LDN-73794, Y-27632, Compound 4, CZC-54252, CZC-25146, Go6976, K252b, LRRK2-in-1, H-1152, sunitinib, K252a, or derivatives and analogs thereof. In some cases, a LRRK2 inhibitor is DNL210, DNL151, or derivatives or analogs thereof. Example LRRK2 inhibitors are shown in Table 1. A LRRK2 inhibitor may be administered in combination with a dopamine receptor stimulating therapy.

TABLE 1

| Compound | |
|---|---|
| Name | Chemical Structure |
| PD-98059 | 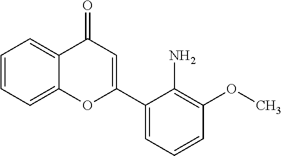 [SMILES: COc1cccc(-c2cc(=O)c3ccccc3o2)c1N] |
| U-0126 | 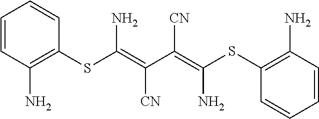 [SMILES: N#CC(=C(/N)Sc1ccccc1N)/C(C#N)=C(\N)Sc1ccccc1N] |
| SB-203580 | 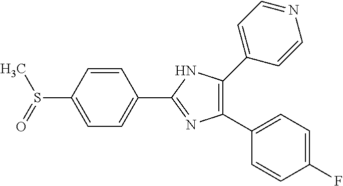 [SMILES: CS(=O)c1ccc(-c2nc(-c3ccc(F)cc3)c(-c3ccncc3)[nH]2)cc1] |
| H-7 | 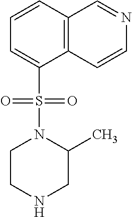 [SMILES: CC1CNCCN1S(=O)(=O)c1cccc2cnccc12] |
| H-9 | 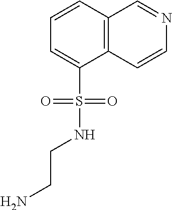 [SMILES: NCCNS(=O)(=O)c1cccc2cnccc12] |
| Staurosporine | 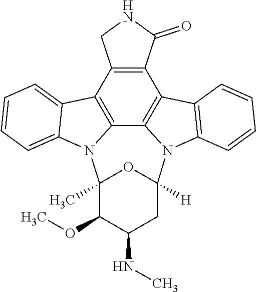 [SMILES: CN[C@@H]1C[C@H]2O[C@@](C)([C@@H]1OC)n1c3ccccc3c3c4c(c5c6ccccc6n2c5c31)C(=O)NC4] |
| AG-494 | 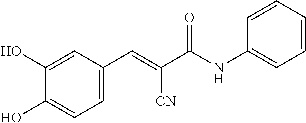 [SMILES: N#C/C(=C\c1ccc(O)c(O)c1)C(=O)Nc1ccccc1] |
| 5-iodo- tubercidin | 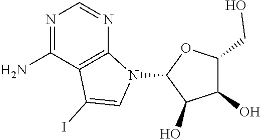 [SMILES: Nc1ncnc2c1c(I)cn2[C@@H]1O[C@H](CO)[C@@H](O)[C@H]1O] |
| LFM-A13 | 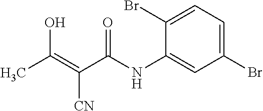 [SMILES: C/C(O)=C(\C#N)C(=O)Nc1cc(Br)ccc1Br] |
| SB-202190 | 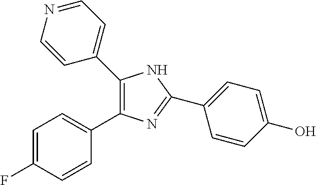 [SMILES: Oc1ccc(-c2nc(-c3ccc(F)cc3)c(-c3ccncc3)[nH]2)cc1] |
| PP2 | 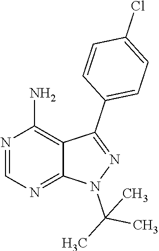 [SMILES: CC(C)(C)n1nc(-c2ccc(Cl)cc2)c2c(N)ncnc21] |
| ZM336372 | 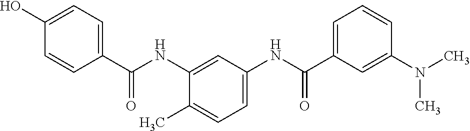 [SMILES: Cc1ccc(NC(=O)c2cccc(N(C)C)c2)cc1NC(=O)c1ccc(O)cc1] |
| SU4312 | 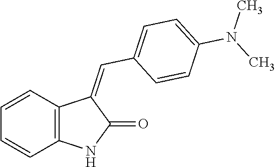 [SMILES: CN(C)c1ccc(/C=C2\C(=O)Nc3ccccc32)cc1] |
| AG-1296 | 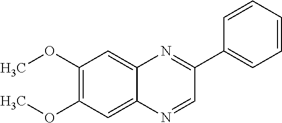 [SMILES: COc1cc2ncc(-c3ccccc3)nc2cc1OC] |
| AG-825 | 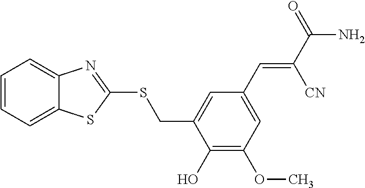 [SMILES: COc1cc(/C=C(\C#N)C(N)=O)cc(CSc2nc3ccccc3s2)c1O] |
| Lavendustin A | 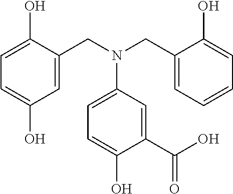 [SMILES: O=C(O)c1cc(N(Cc2ccccc2O)Cc2cc(O)ccc2O)ccc1O] |
| RG-14620 | 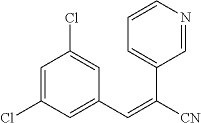 [SMILES: N#C/C(=C/c1cc(Cl)cc(Cl)c1)c1cccnc1] |
| Tyrphostin 23 | 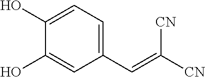 [SMILES: N#CC(C#N)=Cc1ccc(O)c(O)c1] |
| Tyrphostin 25 | 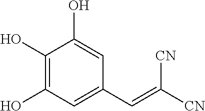 [SMILES: N#CC(C#N)=Cc1cc(O)c(O)c(O)c1] |
| Tyrphostin 46 | 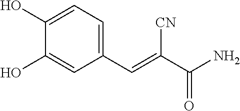 [SMILES: N#C/C(=C\c1ccc(O)c(O)c1)C(N)=O] |
| Tyrphostin 47 | 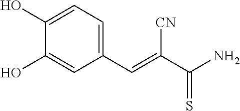 [SMILES: N#C/C(=C\c1ccc(O)c(O)c1)C(N)=S] |
| GW5074 | 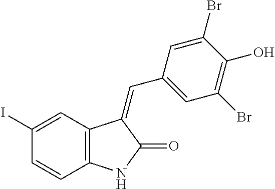 [SMILES: O=C1Nc2ccc(I)cc2/C1=C/c1cc(Br)c(O)c(Br)c1] |
| Palmitoyl-DL- carnitine Cl | 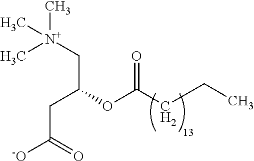 |
| Rottlerin | 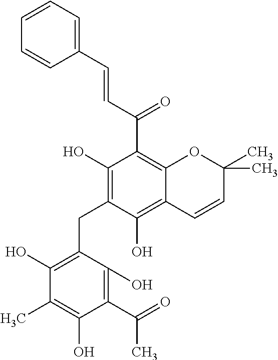 [SMILES: CC(=O)c1c(O)c(C)c(O)c(Cc2c(O)c3c(c(C(=O)/C=C/c4ccccc4)c2O)OC(C)(C)C=C3)c1O] |
| Genistein | 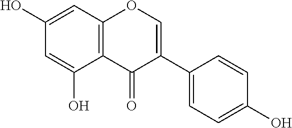 [SMILES: O=c1c(-c2ccc(O)cc2)coc2cc(O)cc(O)c12] |
| Daidzein | 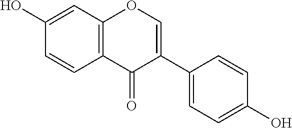 [SMILES: O=c1c(-c2ccc(O)cc2)coc2cc(O)ccc12] |
| Erbstatin analog | 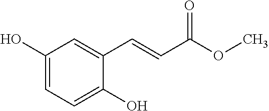 |
| Quercetin dihydrate | 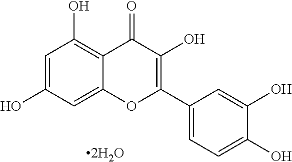 [SMILES: O=c1c(O)c(-c2ccc(O)c(O)c2)oc2cc(O)cc(O)c12] |
| Tyrphostin 51 | 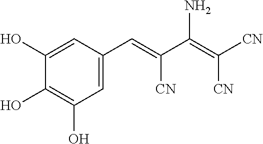 [SMILES: N#CC(C#N)=C(N)/C(C#N)=C/c1cc(O)c(O)c(O)c1] |
| Tyrphostin 1 | 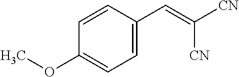 |
| Tyrphostin AG1288 | 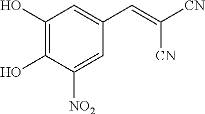 [SMILES: N#CC(C#N)=Cc1cc(O)c(O)c([N+](=O)[O-])c1] |
| Tyrphostin AG1478 | 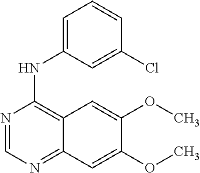 [SMILES: COc1cc2ncnc(Nc3cccc(Cl)c3)c2cc1OC] |
| Tyrphostin AG1295 | 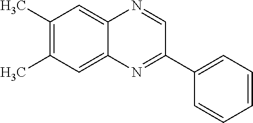 [SMILES: Cc1cc2ncc(-c3ccccc3)nc2cc1C] |
| Tyrphostin 9 | 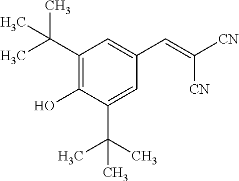 [SMILES: CC(C)(C)c1cc(C=C(C#N)C#N)cc(C(C)(C)C)c1O] |
| Hydroxy-2- naphthalene ethylphosphoric acid | 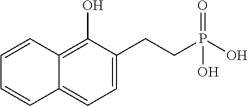 [SMILES: O=P(O)(O)CCc1ccc2ccccc2c1O] |
| SU1498 | 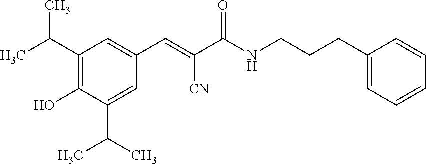 [SMILES: CC(C)c1cc(/C=C(\C#N)C(=O)NCCCc2ccccc2)cc(C(C)C)c1O] |
| ZM449829 | 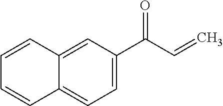 |
| BAY11-7082 | 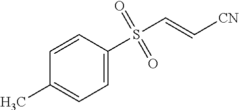 |
| 5,6-dichloro-1- beta-D- ribofutanosyl- benzimidiazole | 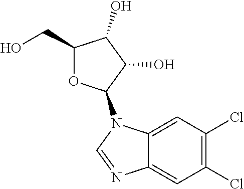 [SMILES: OC[C@@H]1O[C@H](n2cnc3cc(Cl)c(Cl)cc32)[C@@H](O)[C@H]1O] |
| 2,2′,3,3′,4,4′- hexahydroxy- 1,1′-biphenyl- 6,6′- dimenthanol dimethyl ester | 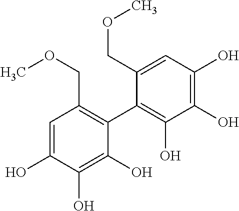 [SMILES: COCc1cc(O)c(O)c(O)c1-c1c(COC)cc(O)c(O)c1O] |
| SP600125 | 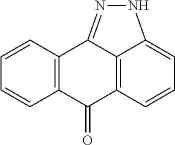 [SMILES: O=C1c2ccccc2-c2n[nH]c3cccc1c23] |
| Indirubin | 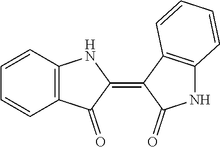 [SMILES: O=C1Nc2ccccc2/C1=C1\Nc2ccccc2C1=O] |
| Damnacanthal | 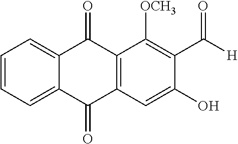 [SMILES: COc1c(C=O)c(O)cc2c1C(=O)c1ccccc1C2=O] |
| Piceatannol | 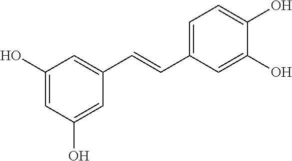 [SMILES: Oc1cc(O)cc(/C=C/c2ccc(O)c(O)c2)c1] |
| PP1 | 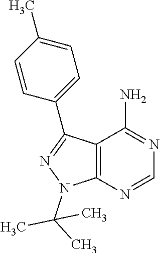 [SMILES: Cc1ccc(-c2nn(C(C)(C)C)c3ncnc(N)c23)cc1] |
| AG-490 | 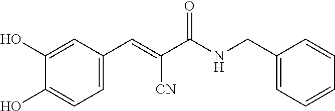 [SMILES: N#C/C(=C\c1ccc(O)c(O)c1)C(=O)NCc1ccccc1] |
| AG-126 | 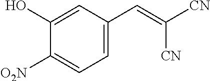 [SMILES: N#CC(C#N)=Cc1ccc([N+](=O)[O-])c(O)c1] |
| AG-370 | 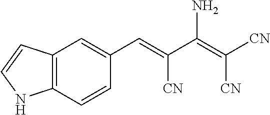 [SMILES: N#CC(C#N)=C(N)/C(C#N)=C/c1ccc2[nH]ccc2c1] |
| AG-879 | 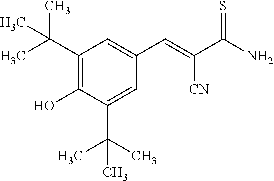 [SMILES: CC(C)(C)c1cc(/C=C(\C#N)C(N)=S)cc(C(C)(C)C)c1O] |
| Indirubin-3- monooxine | 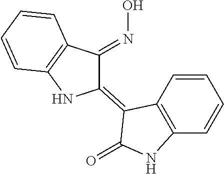 [SMILES: O=C1Nc2ccccc2/C1=C1/Nc2ccccc2/C1=N\O] |
| Cantharidic acid | 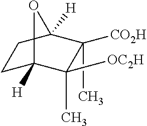 |
| Cantharidin | 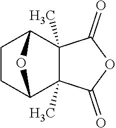 |
| Endothall | 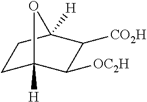 |
| Benzyl- phosphoric acid | 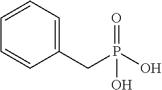 [SMILES: O=P(O)(O)Cc1ccccc1] |
| L-p-bromo- tetraamisole oxalate | 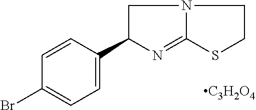 |
| RK-682 | 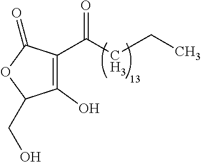 |
| LY294002 | 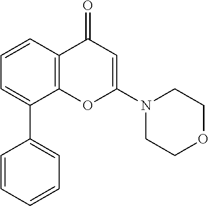 [SMILES: O=c1cc(N2CCOCC2)oc2c(-c3ccccc3)cccc12] |
| Wortmannin | 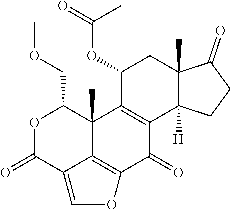 [SMILES: COC[C@H]1OC(=O)c2coc3c2[C@@]1(C)C1=C(C3=O)[C@@H]2CCC(=O)[C@@]2(C)C[C@H]1OC(C)=O] |
| GP109203X | 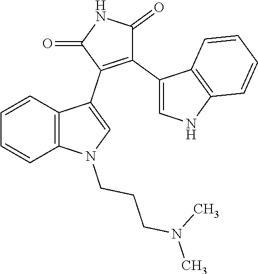 [SMILES: CN(C)CCCn1cc(C2=C(c3c[nH]c4ccccc34)C(=O)NC2=O)c2ccccc21] |
| Hypercin | 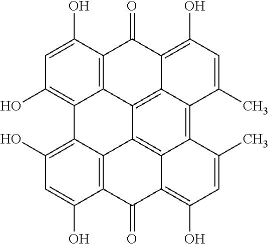 [SMILES: Cc1cc(O)c2c(=O)c3c(O)cc(O)c4c5c(O)cc(O)c6c(=O)c7c(O)cc(C)c8c1c2c(c34)c(c78)c65] |
| Ro31-8220 | 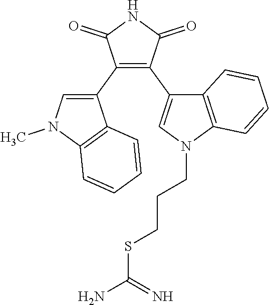 [SMILES: Cn1cc(C2=C(c3cn(CCCSC(=N)N)c4ccccc34)C(=O)NC2=O)c2ccccc21] |
| Sphingosine | 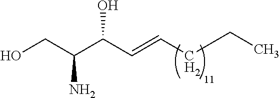 |
| RWJ-60475 | 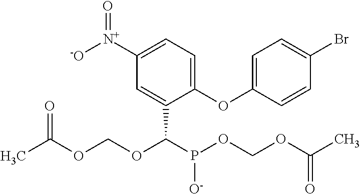 |
| Levarmisole HCl | 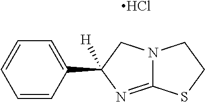 [SMILES: Cl ; c1ccc([C@H]2CN3CCSC3=N2)cc1] |
| Tetramisole HCl | 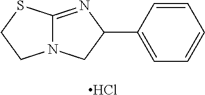 [SMILES: c1ccc(C2CN3CCSC3=N2)cc1 ; Cl] |
| Cypermethrin | 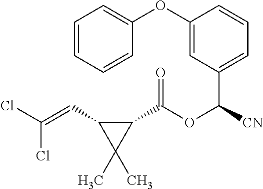 [SMILES: CC1(C)[C@H](C=C(Cl)Cl)[C@@H]1C(=O)O[C@H](C#N)c1cccc(Oc2ccccc2)c1] |
| Deltamethrin | 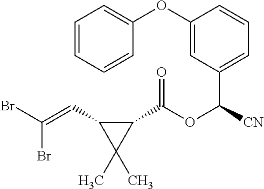 [SMILES: CC1(C)[C@H](C=C(Br)Br)[C@@H]1C(=O)O[C@H](C#N)c1cccc(Oc2ccccc2)c1] |
| Fenvaierate | 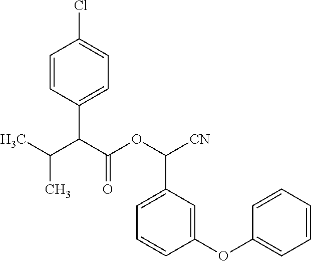 [SMILES: CC(C)C(C(=O)OC(C#N)c1cccc(Oc2ccccc2)c1)c1ccc(Cl)cc1] |
| H-89 | 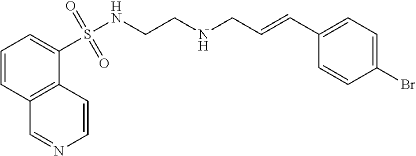 [SMILES: O=S(=O)(NCCNC/C=C/c1ccc(Br)cc1)c1cccc2cnccc12] |
| H-8 | 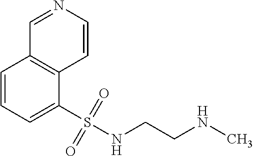 [SMILES: CNCCNS(=O)(=O)c1cccc2cnccc12] |
| HA-1004 | 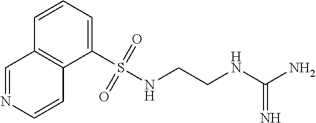 [SMILES: N=C(N)NCCNS(=O)(=O)c1cccc2cnccc12] |
| HA-1077 | 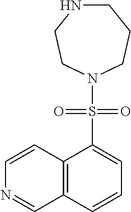 [SMILES: O=S(=O)(c1cccc2cnccc12)N1CCCNCC1] |
| 2-hydroxy-5- (2,5- dihydroxybenzyl- amino) benzoic acid | 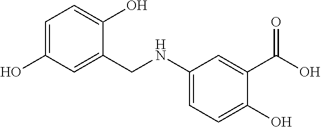 [SMILES: O=C(O)c1cc(NCc2cc(O)ccc2O)ccc1O] |
| KN-62 | 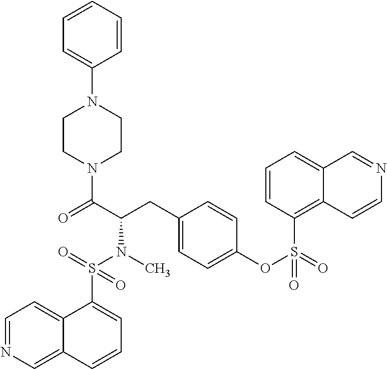 [SMILES: CN([C@@H](Cc1ccc(OS(=O)(=O)c2cccc3cnccc23)cc1)C(=O)N1CCN(c2ccccc2)CC1)S(=O)(=O)c1cccc2cnccc12] |
| KN-93 | 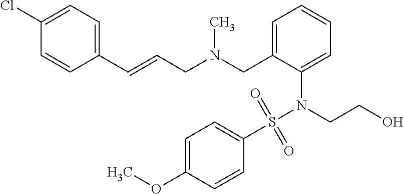 [SMILES: COc1ccc(S(=O)(=O)N(CCO)c2ccccc2CN(C)C/C=C/c2ccc(Cl)cc2)cc1] |
| Tyrphostin 8 | 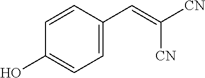 [SMILES: N#CC(C#N)=Cc1ccc(O)cc1] |
| Cinngel | 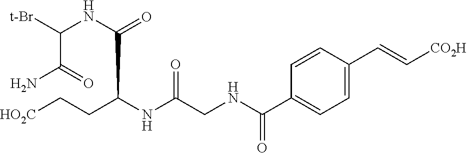 |
| LDN-22684 | 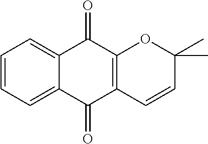 [SMILES: CC1(C)C=CC2=C(O1)C(=O)c1ccccc1C2=O] |
| LDN-73794 | 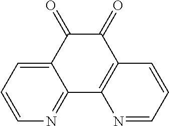 [SMILES: O=C1C(=O)c2cccnc2-c2ncccc21] |
| Y-27632 | 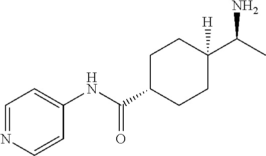 [SMILES: C[C@H](N)[C@H]1CC[C@H](C(=O)Nc2ccncc2)CC1] |
| Compound 4 | 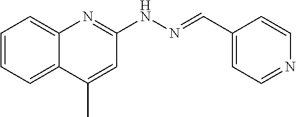 [SMILES: Cc1cc(N/N=C/c2ccncc2)nc2ccccc12] |
| CZC-54252 | 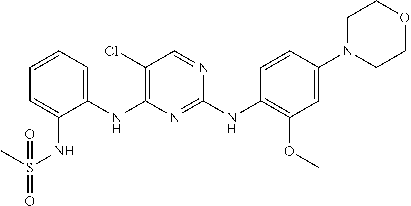 [SMILES: COc1cc(N2CCOCC2)ccc1Nc1ncc(Cl)c(Nc2ccccc2NS(C)(=O)=O)n1] |
| ML-7 | 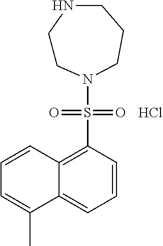 [SMILES: Cc1cccc2c(S(=O)(=O)N3CCCNCC3)cccc12 ; Cl] |
| ML-9 | 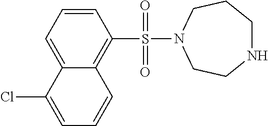 [SMILES: O=S(=O)(c1cccc2c(Cl)cccc12)N1CCCNCC1] |
| 2-aminopurine | 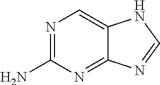 |
| N9-isopropyl olomoucine | 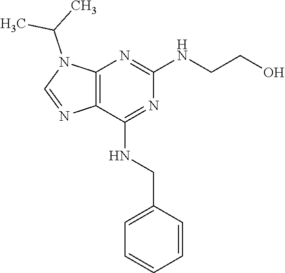 [SMILES: CC(C)n1cnc2c(NCc3ccccc3)nc(NCCO)nc21] |
| Olomoucine | 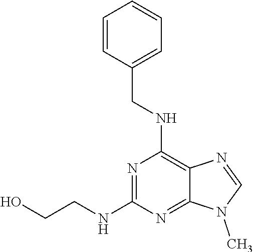 [SMILES: Cn1cnc2c(NCc3ccccc3)nc(NCCO)nc21] |
| Iso-olomoucine | 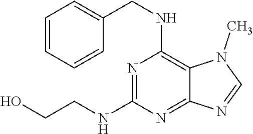 [SMILES: Cn1cnc2nc(NCCO)nc(NCc3ccccc3)c21] |
| CZC-25146 | 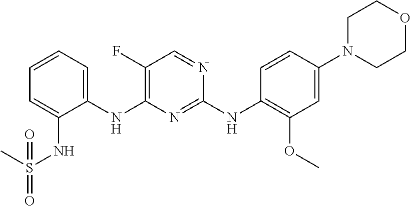 [SMILES: COc1cc(N2CCOCC2)ccc1Nc1ncc(F)c(Nc2ccccc2NS(C)(=O)=O)n1] |
| Go6976 | 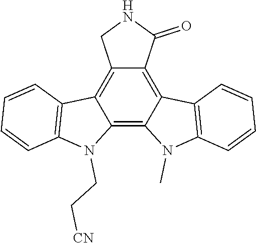 [SMILES: Cn1c2ccccc2c2c3c(c4c5ccccc5n(CCC#N)c4c21)CNC3=O] |
| K252b | 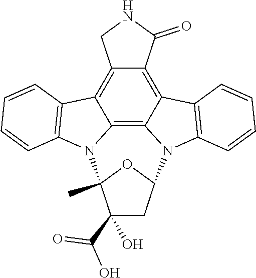 [SMILES: C[C@@]12O[C@@H](C[C@@]1(O)C(=O)O)n1c3ccccc3c3c4c(c5c6ccccc6n2c5c31)CNC4=O] |
| LRRK2-in-1 | 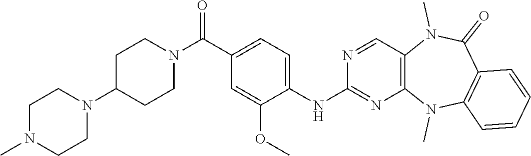 [SMILES: COc1cc(C(=O)N2CCC(N3CCN(C)CC3)CC2)ccc1Nc1ncc2c(n1)N(C)c1ccccc1C(=O)N2C] |
| H-1152 | 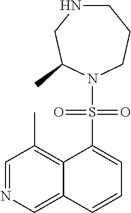 [SMILES: Cc1cncc2cccc(S(=O)(=O)N3CCCNC[C@@H]3C)c12] |
| Sunitinib | 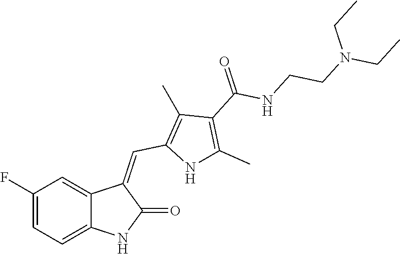 [SMILES: CCN(CC)CCNC(=O)c1c(C)[nH]c(/C=C2\C(=O)Nc3ccc(F)cc32)c1C] |
| Roscovitine | 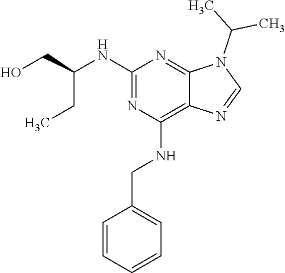 [SMILES: CC[C@@H](CO)Nc1nc(NCc2ccccc2)c2ncn(C(C)C)c2n1] |
| K252a | 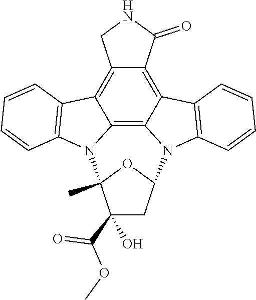 [SMILES: COC(=O)[C@]1(O)C[C@@H]2O[C@@]1(C)n1c3ccccc3c3c4c(c5c6ccccc6n2c5c31)C(=O)NC4] |

### Protein Kinase Inhibitors

Multiple small molecule kinase inhibitors have been approved by US FDA and are available in the market, including imatinib (Gleevec), sorafenib (Nexavar), sunitinib (Sutent), rapamycin (Sirolimus) to name a few. Potential druggable kinase-related signaling pathways include protein kinase Cd, the MLK-cjun N-terminal kinase (JNK) signaling cascade, and AKT/protein kinase B (PKB) signaling cascade, all of which are kinases implicated in programmed cell death. CEP1347, an MLK inhibitor has been shown to have neuroprotective effects in a variety of neurodegenerative models. One or more protein kinase inhibitors disclosed herein may be used as a therapy to treat a neurological disease, for example Parkinson's disease. Efficacy of a protein kinase inhibitor may be evaluated using the methods disclosed herein.

### Anticholinergic Therapeutics

An anticholinergic therapeutic may be administered to treat neurodegenerative diseases. An anticholinergic therapeutic may be administered to treat synucleinopathies (e.g., Parkinson's disease, MSA, or Lewy body dementia). Efficacy of an anticholinergic therapeutic may be evaluated using the methods disclosed herein. An anticholinergic therapeutic may be trihexyphenidyl, benztropine, orphenadrine, procyclidine, biperiden, or derivatives and analogs thereof.

### Adenosine Receptor Antagonists

An adenosine receptor antagonist may be administered to treat neurodegenerative diseases. An adenosine receptor antagonist may be administered to treat synucleinopathies (e.g., Parkinson's disease, MSA, or Lewy body dementia). Efficacy of an adenosine receptor antagonist may be evaluated using the methods disclosed herein. An adenosine receptor antagonist may be istradefylline, preladenant, tozadenant, vipadenant or derivatives and analogs thereof.

### Glutamate Receptor Antagonists

A glutamate receptor antagonist may be administered to treat neurodegenerative diseases. A glutamate receptor antagonist may be administered to treat synucleinopathies (e.g., Parkinson's disease, MSA, or Lewy body dementia). Efficacy of a glutamate receptor antagonist may be evaluated using the methods disclosed herein. A glutamate receptor antagonist may be istradefylline, preladenant, tozadenant, vipadenant or derivatives and analogs thereof.

### Glutamate Receptor Activators

A glutamate receptor activator may be administered to treat neurodegenerative diseases. A glutamate receptor activator may be administered to treat synucleinopathies (e.g., Parkinson's disease, MSA, or Lewy body dementia). Efficacy of a glutamate receptor activator may be evaluated using the methods disclosed herein. A glutamate receptor activator may be ADX88178 or derivatives and analogs thereof.

### Adrenoceptor Antagonists

An adrenoceptor antagonist may be administered to treat neurodegenerative diseases. An adrenoceptor antagonist may be administered to treat synucleinopathies (e.g., Parkinson's disease, MSA, or Lewy body dementia). Efficacy of an adrenoceptor antagonist may be evaluated using the methods disclosed herein. An adrenoceptor antagonist may be idazoxan, fipamezole, or derivatives and analogs thereof.

### Serotonin Receptor Agonists

A serotonin receptor agonist may be administered to treat neurodegenerative diseases. A serotonin receptor agonist may be administered to treat synucleinopathies (e.g., Parkinson's disease, MSA, or Lewy body dementia). Efficacy of a serotonin receptor agonist may be evaluated using the methods disclosed herein. A serotonin receptor agonist may be sarizotan, piclozotan, or derivatives and analogs thereof.

### Substrate Reduction Therapy

Substrate reduction therapy may be administered to treat neurodegenerative diseases. A substrate reduction therapy may be administered to treat synucleinopathies (e.g., Parkinson's disease, MSA, or Lewy body dementia). Efficacy of a substrate reduction therapy may be evaluated using the methods disclosed herein. A substrate reduction therapy may include inhibition of the enzyme glucosylceramide synthase (GCS) or pharmacological chaperone therapy. Inhibition of GCS may include, but is not limited to, administration of miglustat. Pharmacological chaperone therapy may include, but is not limited to, administration of isofagomine, ambroxol, noninhibitory pyrazolopyrimindines, pyrimethamine, a naphthalimide, NBn-LABNAc, a bisnaphthalimide, nitro-indan-1-one, pyrrolo[3,4-d]pyridazin1-one, or any combination thereof.

### Genetic Enzyme Replacement Therapy

Genetic enzyme replacement therapy may be administered to treat neurodegenerative diseases. A genetic enzyme replacement therapy may be administered to treat synucleinopathies (e.g., Parkinson's disease, MSA, or Lewy body dementia). Efficacy of a genetic enzyme replacement therapy may be evaluated using the methods disclosed herein. Genetic enzyme replacement therapy may include, but is not limited to, administration of a vector comprising aromatic amino acid decarboxylase (AADC), tyrosine hydroxylase (TH), or GTP-cyclohydrolase-1 (GCH1).

### Anti-α Synuclein Antibodies

Anti-α synuclein antibodies may be administered to treat neurodegenerative diseases. Anti-α synuclein antibodies may be administered to treat synucleinopathies (e.g., Parkinson's disease, MSA, or Lewy body dementia). Efficacy of an anti-α synuclein antibody may be evaluated using the methods disclosed herein. Anti-α synuclein antibodies may include, but are not limited to, anti-α synuclein antibody Syn211, Syn303, PRX002/RO7046015, or BIIB054.

### α Synuclein Vaccine

α synuclein vaccines may be administered to treat neurodegenerative diseases. α synuclein vaccines may be administered to treat synucleinopathies (e.g., Parkinson's disease, MSA, or Lewy body dementia). Efficacy of an α synuclein vaccine may be evaluated using the methods disclosed herein. α synuclein vaccines may include, but are not limited to, virus-like particles containing a synuclein peptides or a peptide vaccine, for example using an AFFITOPE® immunogen.

### Neuroprotectants

Neuroprotectants may be administered to treat neurodegenerative diseases. A neuroprotectant may be administered to treat synucleinopathies (e.g., Parkinson's disease, MSA, or Lewy body dementia). Efficacy of a neuroprotectant may be evaluated using the methods disclosed herein. Neuroprotectants may include c-Abl inhibitors, parkin modulators, neuroinflammation modulators, or modified fatty-acid therapeutics such as RT-001 (Retrotope Pharmaceuticals, Los Altos, Calif.). c-Abl inhibitors may include, but are not limited to, bosutinib, imatinib, nilotinib, 1-naphthyl PP1, bcr-abl inhibitor, dasatinib (monohydrate), bcr-abl inhibitor II, PP121, herbimycin A, dasatinib, DCC-2036, or AT9283. Parkin modulators may include, but are not limited to, AF-6. Neuroinflammation modulators may include, but are not limited to, alpha-melanocyte-stimulating hormone (MSH-α), vasoactive intestinal peptide (VIP), neuropeptide Y (NPY), des-acyl-ghrelin (DAG), or acyl-ghrelin (AG).

### Neurological Disorders

“Neurological disorders,” as used herein, include Parkinson's disease, Multisystem Lewy body disease (MLBD), parkinsonism, dementia with Lewy bodies (DLB), pure autonomic failure (PAF), Parkinson's disease dementia (PDD), multiple system atrophy, progressive supranuclear palsy, corticobasal degeneration, fronto-temporal dementia, Alzheimer's disease without Parkinson's disease, atypical parkinsonism, α-synuclein- or tau-related neuropathy, Lewy neurites or neuronal cell loss in substantia nigra, and α-synuclein-positive Lewy bodies. Neurological disorders can comprise movement disorders (e.g., movement disorders caused by a neurological disorder). A neurological disorder may be Parkinson's disease or multiple system atrophy (MSA). In some cases, a neurological disorder is Parkinson's disease.

### Movement Disorders

Methods described herein can be used to evaluate severity or progression of a movement disorder. Movement disorders can comprise symptoms such as tremors, muscle rigidity, slow movement, and postural instability. Movement disorders may be caused by a neurological disorder. In some instances, movement disorders include, but are not limited to, Parkinson's disease, MSA, progressive supranuclear palsy, viral parkinsonism, essential tremor, toxin-induced parkinsonism, arteriosclerotic parkinsonism, Parkinsonism-dementia complex of Guam, and normal pressure hydrocephalus (NPH). In some instances, treatment with certain drugs may induce movement disorder symptoms. Drugs that induce movement disorder symptoms may include, but are not limited to, chlorpromazine (Thorazine), haloperidol (Haldol), metoclopramide (Reglan), and valproate (Depacon).

### Subject

The methods disclosed herein may be used to evaluate a subject. A subject may include for example, a male or female adult, child, newborn, or fetus. A subject may be any target of therapeutic administration. A subject may be a test subject or a reference subject. A subject may be associated with a condition or disease or disorder, asymptomatic or symptomatic, have increased or decreased susceptibility to a disease or disorder, be associated or unassociated with a treatment or treatment regimen, or any combination thereof. As used in the present disclosure, a cohort may represent an ethnic group, a patient group, a particular age group, a group not associated with a particular disease or disorder, a group associated with a particular disease or disorder, a group of asymptomatic subjects, a group of symptomatic subjects, or a group or subgroup of subjects associated with a particular response to a treatment regimen or clinical trial. A subject may be suffering from a disorder, for example, a neurological disorder. A subject may be a test subject, a patient, or a candidate being treated with one or more therapeutics, wherein the subject, patient, or candidate is evaluated for treatment efficacy by one or more methods of the present disclosure. A subject may be evaluated using a Timed Up and Go test, as described herein.

Subjects of all ages are contemplated in the present disclosure. Subjects may be from specific age subgroups, such as those over the age of 1, over the age of 2, over the age of 3, over the age of 4, over the age of 5, over the age of 6, over the age of 7, over the age of 8, over the age of 9, over the age of 10, over the age of 15, over the age of 20, over the age of 25, over the age of 30, over the age of 35, over the age of 40, over the age of 45, over the age of 50, over the age of 55, over the age of 60, over the age of 65, over the age of 70, over the age of 75, over the age of 80, or over the age of 85. Other embodiments of the disclosure pertain to other age groups, such as subjects aged less than age 85, less than age 80, less than age 75, less than age 70, less than age 65, less than age 60, less than age 55, less than age 50, less than age 45, less than age 40, less than age 35, less than age 30, less than age 25, less than age 20, less than age 15, less than age 10, less than age 9, less than age 8, less than age 6, less than age 5, less than age 4, less than age 3, less than age 2, or less than age 1. Other embodiments relate to subjects with age at onset of the disease in any of particular age or age ranges defined by the numerical values described in the above or other numerical values bridging these numbers. It is also contemplated that a range of ages may be relevant in certain embodiments, such as age at onset at more than age 15 but less than age 20. Other age ranges are however also contemplated, including all age ranges bracketed by the age values listed in the above.

### EXAMPLES

### Example 1—Method for Performing the Timed Up and go Test

A subject begins seated in a chair. The subject stands from the chair, walks forward to a position three meters from the front of the chair, turns around and walks back to the chair, and sits down in the chair. An additional individual monitors the subject and measures the time the subject takes to perform the series of tasks. This measurement of time is recorded as the result of the Timed Up and Go test.

### Example 2—Study of Efficacy and Safety of a LRRK2 Inhibitor as an Add-on Therapy in Patients with Parkinson's Disease

### Study Description

The purpose of this study is to determine the long-term efficacy and safety of a LRRK2 inhibitor, compared to placebo, as add-on therapy in patients with idiopathic Parkinson's disease with motor fluctuations, who are currently receiving a stable dose of levodopa.

### Arms and Interventions

| Arm | Intervention/treatment |
|---|---|
| Experimental | Drug: LRRK2 inhibitor |
| Placebo Comparator | Drug: Placebo |

### Outcome Measures

### Primary Outcome Measures

Mean change in time to perform the Timed Up and Go test from baseline to endpoint

### Secondary Outcome Measures

Mean change in the dyskinesias rating scale (DRS) during “on” time from baseline to endpoint; mean change in total score for unified Parkinson's disease rating scale (UPDRS) IV; mean change in ON time.

### Example 3—Performing the Timed Up and go Test to Evaluate the Efficacy of a LRRK2 Inhibitor in a Subject with Parkinson's Disease

A subject suffering from Parkinson's disease is treated with L-dopa, thereby improving the symptoms of bradykinesia and rigidity in the subject. Following this, the Timed Up and Go test is performed on the subject to obtain a first measurement of time. Next, the individual is treated with a therapeutically effective amount of a LRRK2 inhibitor in addition to L-dopa. Statistically significant improvements in baradykinesia and rigidity in the patient are not observed in the treatment with both the LRRK2 inhibitor and L-dopa in combination as compared with the use of L-dopa alone. Next, the Timed Up and Go test is performed on the subject to obtain a second measurement of time. A significant reduction is observed in the second measurement of time relative to the first measurement of time, thereby indicating the efficacy of the LRRK2 inhibitor in treating Parkinson's disease independent of the efficacy of L-dopa treatment.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method of evaluating an efficacy of treatment for a neurological disorder comprising:
(a) providing a subject suffering from a neurological disorder with a dopamine receptor stimulating therapy;
(b) performing a Timed Up and Go test on the subject to obtain a first measurement;
(c) providing the subject with an additional therapeutic;
(d) performing a Timed Up and Go test on the subject to obtain a second measurement; and
(e) determining the efficacy of the additional therapeutic based on the difference between the first measurement and the second measurement, wherein the efficacy of the therapeutic is determined independently from the efficacy of the dopamine receptor stimulating therapy.

2. The method of claim 1, wherein the first and second measurements are measurements of time.

3. (canceled)

4. The method of claim 1, wherein the additional therapeutic is determined to be effective if the second measurement is not increased or reduced relative to the first measurement.

5. (canceled)

6. (canceled)

7. (canceled)

8. The method of claim 1, wherein the subject suffering from a neurological disorder is further treated with the additional therapeutic if the second measurement is not increased or reduced relative to the first measurement.

9. (canceled)

10. A method of evaluating the progression of a neurological disorder comprising:
(a) providing the subject with a therapeutic regimen comprising a dopamine receptor stimulating therapy and an additional therapeutic;
(b) performing a Timed Up and Go test on the subject; and
(c) evaluating the progression of the neurological disorder based on the Timed Up and Go test.

11. The method of claim 10, further comprising modifying the dose of the therapeutic regimen or providing the subject with a further additional therapeutic based on the evaluating in (c).

12. (canceled)

13. A method of determining an optimum dosage level of a therapeutic for treatment of a neurological disorder in a subject suffering therefrom, comprising:
(a) administering to the subject a first dosage level of a therapeutic agent for a number of doses at a dosing frequency;
(b) performing a Timed Up and Go test to the subject to obtain a first measurement;
(c) administering an adjusted dosage level of the therapeutic agent, relative to the first dosage level, for a number of doses at a dosing frequency;
(d) performing a second Timed Up and Go test to the subject to obtain a second measurement; and
(e) determining an optimal dosage level from the two dosage levels administered, based on performance in Timed Up and Go test and initiation or abatement of side effects.

14. The method of claim 13, further comprising repeating (b)-(e), wherein the adjusted dosage level is an increased dosage level until a maximum dosage level is achieved, no further improvement in test performance is detected, side effects outweigh benefits of treatment, or a combination thereof.

15. (canceled)

16. The method of claim 13, further comprising repeating (b)-(e), wherein the adjusted dosage level is a decreased dosage level until a minimum dosage level is achieved, test performance deteriorates, side effects are minimized, or a combination thereof.

17. The method of claim 1, wherein the neurological disorder is multiple system atrophy, Parkinson's disease or a movement disorder.

18. (canceled)

19. The method of claim 1, wherein the additional therapeutic is a LRRK2 inhibitor, an anticholinergic therapeutic, an adenosine receptor antagonist, a glutamate receptor antagonist, a glutamate receptor activator, an adrenoceptor antagonist, or a serotonin receptor agonist.

20. (canceled)

21. (canceled)

22. The method of claim 19, wherein the anticholinergic therapeutic is selected from the group consisting of trihexyphenidyl, benztropine, orphenadrine, procyclidine, biperiden, and derivatives and analogs thereof.

23. The method of claim 19, wherein the adenosine receptor antagonist is selected from the group consisting of istradefylline, preladenant, tozadenant, vipadenant and derivatives and analogs thereof.

24. The method of claim 19, wherein the glutamate receptor antagonist is selected from the group consisting of amantadine, dipraglurant, mavoglurant and derivatives and analogs thereof.

25. The method of claim 19, wherein the glutamate receptor activator is selected from the group consisting of ADX88178 and derivatives and analogs thereof.

26. The method of claim 19, wherein the adrenoceptor antagonist is selected from the group consisting of idazoxan, fipamezole, and derivatives and analogs thereof.

27. The method of claim 19, wherein the serotonin receptor agonist is selected from the group consisting of sarizotan, piclozotan, and derivatives and analogs thereof.

28. The method of claim 1, wherein the dopamine receptor stimulating therapy comprises L-dopa, levodopa, levodopa and carbidopa, a dopamine agonist, a monoamine oxidase B inhibitor, a catechol-O-methyltransferase inhibitor, or any combination thereof.

29.-33. (canceled)
